SEQUENCE LISTING (43 sequences)

SEQ ID NO: 1 - DNA, 4394 bp, Homo sapiens
attacgattcttatgtgtgtgtgatatttaaagaaatgtgaaaatcccttttcacccttttcagtgtctagggagccagatttctttccgtctgttaatatataatacaatttctcacaaatatgaaagacccggtcttcaggttctctaaaataatttactgtgtcaagttttgataatattcctagctctctgaaaatgattgaatcaaataagtgtctatttttttttctgcaaacactacccgccagaacaatttccggttgttaaatagtaaagtcaccgttcctttggtaaggaatatttaaaggaactcccttggaaatgaattttgtaataggtgatttttattctgtttttactctatgtgatactgatccttgcactggatttcagtttggcaatatgctttttaaaactgggagtcttaaggtgactggatattagcatttttcactaagttttttggatttgaaagtaccttttgatattatttcttggtgaggctaaaaaaattgataaatagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaaaagatgagaaaaccatacaggagataaaagatgagttatatacatagaaaatgtctcataaatacctgaaatatgttatactttcaaaagcaggcatcaaaagggtatataaatgctatgatctaactttgttaacaaaaaattataaaactacagcaaataaccatgaagatttatagaaaacagaattagaagtaaatacagtaatatttaacagggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaacccatttgtcctgtttaattaggaaataaagacggtagagctagtcggaaaatgaatagttaatcgtggaggaggagaaaacatagctcagtactttgactagaggcagctgggaaagtgacgtcctgtagcatttgctgttctagaaagtacagagacacgtagtgaaaatgggaggatctagaaggaggctgtctcctgtgtagtgtatatttatctgtaagtgagccgttggggaaggattgaatacaggagacgctgtctgcttgctgccttaagacagctagctgaattgctgattaacttttaaaatacccagcttggtttatttttcttagaatctgttgctaagactggggacgctgttttcttttacaaagggaaatctaagttaatttcaaggcattcgaaatggggaaagactattattgcattttgggaattgagaaaggagcttcagatgaagatattaaaaaggcttaccgaaaacaagccctcaaatttcatccggacaagaacaaatctcctcaggcagaggaaaaatttaaagaggtcgcagaagcttatgaagtattgagtgatcctaaaaagagagaaatatatgatcagtttggggaggaagggttgaaaggaggagcaggaggtactgatggacaaggaggtaccttccggtacacctttcatggcgatcctcatgctacatttgctgcatttttcggagggtccaacccctttgaaattttctttggaagacgaatgggtggtggtagagattctgaagaaatggaaatagatggtgatccttttagtgcctttggtttcagcatgaatggatatccaagagacaggaattctgtggggccatcccgcctcaaacaagatcctccagttattcatgaacttagagtatcacttgaagagatatatagtggttgtaccaaacggatgaagatttctcgaaaaaggctaaacgctgatggaaggagttacagatctgaggacaaaattcttaccattgagattaaaaaagggtggaaagaaggcaccaaaattacttttccaagagaaggagatgaaacaccaaatagtattccagcagacattgtttttatcattaaagacaaagatcatccaaaatttaaaagggatggatcaaatataatttatactgctaaaattagtttacgagaggcattgtgtggctgctcaattaatgtaccaacactggatggaagaaacatacctatgtcagtaaatgatattgtgaaacccggaatgaggagaagaattattggatatgggctgccatttccaaaaaatcctgaccaacgtggtgaccttctaatagaatttgaggtgtccttcccagatactatatcttcttcatccaaagaagtacttaggaaacatcttcctgcctcatagaatgaagaactttgttacacatattttgataaggcactgaaaatataaaaggactggtagtttactgatgtagatgtgaattctgtataaagatgtgtaaattcttttgagggttcattaaattgcatgaatagagacgggtcaaataaataggcaaaagggatttttacagttagagataaaagagaaaaccattcactgtattttatttcatttctcctgattcagatatttttagtaatttgcttatatgtaaaagttgtttttgtggagtcagtggatatatttctaatgaagtgctagactatccaattacttaatttcttatacctttagataatcagtatgaaaagttcccatttataatggaaatgaaaattcttaactaaactatacatgtaatatgtatttctagaagagaataaaaacccaagtcagttattagatttaaatcaccttctgaaatgctgctatagggctggtatctgtaaaagaatatcctgatgcatctgtttcaccattttgatttttaaagtatgctgtagcatttcttaataacatcgttgtgatgttcttaaggcagatctttcttcataaaaaggaaagtaatggcaatttctctcctgtggaaatcccaattgcttgaattactgatattttagaatagactttttaaaatgccatatgtaattttatgcaagttgactatatatcttgtacttaataaattataggctcattttgttctctgctagtttaaagtaattcgtttaataatagatgtgtttttagaggaaatgctgttacttggaattaattttccagttatacagtcttctataacttactaataatattctatatgtactttatgtaatttccctaaaaagaatgaactaccactacactatggtgttaaaccaaaatatagggaaaataaacactaactgctgcttatggataatgttgcaactacttgttatgcatataaatattttactttttcacatgtatagattgcatttcttaggtgttttaattttttaaatatatttatgttttaaaaaaaaaaaaaaa

SEQ ID NO: 2 - DNA, 2303 bp, Homo sapiens
attacgattcttatgtgtgtgtgatatttaaagaaatgtgaaaatcccttttcacccttttcagtgtctagggagccagatttctttccgtctgttaatatataatacaatttctcacaaatatgaaagacccggtcttcaggttctctaaaataatttactgtgtcaagttttgataatattcctagctctctgaaaatgattgaatcaaataagtgtctatttttttttctgcaaacactacccgccagaacaatttccggttgttaaatagtaaagtcaccgttcctttggtaaggaatatttaaaggaactcccttggaaatgaattttgtaataggtgatttttattctgtttttactctatgtgatactgatccttgcactggatttcagtttggcaatatgctttttaaaactgggagtcttaaggtgactggatattagcatttttcactaagttttttggatttgaaagtaccttttgatattatttcttggtgaggctaaaaaaattgataaatagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaaaagatgagaaaaccatacaggagataaaagatgagttatatacatagaaaatgtctcataaatacctgaaatatgttatactttcaaaagcaggcatcaaaagggtatataaatgctatgatctaactttgttaacaaaaaattataaaactacagcaaataaccatgaagatttatagaaaacagaattagaagtaaatacagtaatatttaacagggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaacccatttgtcctgtttaattaggaaataaagacggtagagctagtcggaaaatgaatagttaatcgtggaggaggagaaaacatagctcagtactttgactagaggcagctgggaaagtgacgtcctgtagcatttgctgttctagaaagtacagagacacgtagtgaaaatgggaggatctagaaggaggctgtctcctgtgtagtgtatatttatctgtaagtgagccgttggggaaggattgaatacagagacgctgtctgcttgctgccttaagacagctagctgaattgctgattaacttttaaaatacccagcttggtttatttttcttagaatctgttgctaagactggggacgctgttttcttttacaaagggaaatctaagttaatttcaaggcattcgaaa

SEQ ID NO: 3 - DNA, 3058 bp, Homo sapiens
agggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaacccgagacgctgtctgcttgctgccttaagacagctagctgaattgctgattaacttttaaaatacccagcttggtttatttttcttagaatctgttgctaagactggggacgctgttttcttttacaaagggaaatctaagttaatttcaaggcattcgaaatggggaaagactattattgcattttgggaattgagaaaggagcttcagatgaagatattaaaaaggcttaccgaaaacaagccctcaaatttcatccggacaagaacaaatctcctcaggcagaggaaaaatttaaagaggtcgcagaagcttatgaagtattgagtgatcctaaaaagagagaaatatatgatcagtttggggaggaagggttgaaaggaggagcaggaggtactgatggacaaggaggtaccttccggtacacctttcatggcgatcctcatgctacatttgctgcatttttcggagggtccaacccctttgaaattttctttggaagacgaatgggtggtggtagagattctgaagaaatggaaatagatggtgatccttttagtgcctttggtttcagcatgaatggatatccaagagacaggaattctgtggggccatcccgcctcaaacaagatcctccagttattcatgaacttagagtatcacttgaagagatatatagtggttgtaccaaacggatgaagatttctcgaaaaaggctaaacgctgatggaaggagttacagatctgaggacaaaattcttaccattgagattaaaaaagggtggaaagaaggcaccaaaattacttttccaagagaaggagatgaaacaccaaatagtattccagcagacattgtttttatcattaaagacaaagatcatccaaaatttaaaagggatggatcaaatataatttatactgctaaaattagtttacgagaggcattgtgtggctgctcaattaatgtaccaacactggatggaagaaacatacctatgtcagtaaatgatattgtgaaacccggaatgaggagaagaattattggatatgggctgccatttccaaaaaatcctgaccaacgtggtgaccttctaatagaatttgaggtgtccttcccagatactatatcttcttcatccaaagaagtacttaggaaacatcttcctgcctcatagaatgaagaactttgttacacatattttgataaggcactgaaaatataaaaggactggtagtttactgatgtagatgtgaattctgtataaagatgtgtaaattcttttgagggttcattaaattgcatgaatagagacgggtcaaataaataggcaaaagggatttttacagttagagataaaagagaaaaccattcactgtattttatttcatttctcctgattcagatatttttagtaatttgcttatatgtaaaagttgtttttgtggagtcagtggatatatttctaatgaagtgctagactatccaattacttaatttcttatacctttagataatcagtatgaaaagttcccatttataatggaaatgaaaattcttaactaaactatacatgtaatatgtatttctagaagagaataaaaacccaagtcagttattagatttaaatcaccttctgaaatgctgctatagggctggtatctgtaaaagaatatcctgatgcatctgtttcaccattttgatttttaaagtatgctgtagcatttcttaataacatcgttgtgatgttcttaaggcagatctttcttcataaaaaggaaagtaatggcaatttctctcctgtggaaatcccaattgcttgaattactgatattttagaatagactttttaaaatgccatatgtaattttatgcaagttgactatatatcttgtacttaataaattataggctcattttgttctctgctagtttaaagtaattcgtttaataatagatgtgtttttagaggaaatgctgttacttggaattaattttccagttatacagtcttctataacttactaataatattctatatgtactttatgtaatttccctaaaaagaatgaactaccactacactatggtgttaaaccaaaatatagggaaaataaacactaactgctgcttatggataatgttgcaactacttgttatgcatataaatattttactttttcacatgtatagattgcatttcttaggtgttttaattttttaaatatatttatgttttaaaaaaaaaaaaaaa

SEQ ID NO: 4 - DNA, 808 bp, Homo sapiens
agggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaaccc

SEQ ID NO: 5 - DNA, 2500 bp, Homo sapiens
catttgtcctgtttaattaggaaataaagacggtagagctagtcggaaaatgaatagttaatcgtggaggaggagaaaacatagctcagtactttgactagaggcagctgggaaagtgacgtcctgtagcatttgctgttctagaaagtacagagacacgtagtgaaaatgggaggatctagaaggaggctgtctcctgtgtagtgtatatttatctgtaagtgagccgttggggaaggattgaatacaggagacgctgtctgcttgctgccttaagacagctagctgaattgctgattaacttttaaaatacccagcttggtttatttttcttagaatctgttgctaagactggggacgctgttttcttttacaaagggaaatctaagttaatttcaaggcattcgaaatggggaaagactattattgcattttgggaattgagaaaggagcttcagatgaagatattaaaaaggcttaccgaaaacaagccctcaaatttcatccggacaagaacaaatctcctcaggcagaggaaaaatttaaagaggtcgcagaagcttatgaagtattgagtgatcctaaaaagagagaaatatatgatcagtttggggaggaagggttgaaaggaggagcaggaggtactgatggacaaggaggtaccttccggtacacctttcatggcgatcctcatgctacatttgctgcatttttcggagggtccaacccctttgaaattttctttggaagacgaatgggtggtggtagagattctgaagaaatggaaatagatggtgatccttttagtgcctttggtttcagcatgaatggatatccaagagacaggaattctgtggggccatcccgcctcaaacaagatcctccagttattcatgaacttagagtatcacttgaagagatatatagtggttgtaccaaacggatgaagatttctcgaaaaaggctaaacgctgatggaaggagttacagatctgaggacaaaattcttaccattgagattaaaaaagggtggaaagaaggcaccaaaattacttttccaagagaaggagatgaaacaccaaatagtattccagcagacattgtttttatcattaaagacaaagatcatccaaaatttaaaagggatggatcaaatataatttatactgctaaaattagtttacgagaggcattgtgtggctgctcaattaatgtaccaacactggatggaagaaacatacctatgtcagtaaatgatattgtgaaacccggaatgaggagaagaattattggatatgggctgccatttccaaaaaatcctgaccaacgtggtgaccttctaatagaatttgaggtgtccttcccagatactatatcttcttcatccaaagaagtacttaggaaacatcttcctgcctcatagaatgaagaactttgttacacatattttgataaggcactgaaaatataaaaggactggtagtttactgatgtagatgtgaattctgtataaagatgtgtaaattcttttgagggttcattaaattgcatgaatagagacgggtcaaataaataggcaaaagggatttttacagttagagataaaagagaaaaccattcactgtattttatttcatttctcctgattcagatatttttagtaatttgcttatatgtaaaagttgtttttgtggagtcagtggatatatttctaatgaagtgctagactatccaattacttaatttcttatacctttagataatcagtatgaaaagttcccatttataatggaaatgaaaattcttaactaaactatacatgtaatatgtatttctagaagagaataaaaacccaagtcagttattagatttaaatcaccttctgaaatgctgctatagggctggtatctgtaaaagaatatcctgatgcatctgtttcaccattttgatttttaaagtatgctgtagcatttcttaataacatcgttgtgatgttcttaaggcagatctttcttcataaaaaggaaagtaatggcaatttctctcctgtggaaatcccaattgcttgaattactgatattttagaatagactttttaaaatgccatatgtaattttatgcaagttgactatatatcttgtacttaataaattataggctcattttgttctctgctagtttaaagtaattcgtttaataatagatgtgtttttagaggaaatgctgttacttggaattaattttccagttatacagtcttctataacttactaataatattctatatgtactttatgtaatttccctaaaaagaatgaactaccactacactatggtgttaaaccaaaatatagggaaaataaacactaactgctgcttatggataatgttgcaactacttgttatgcatataaatattttactttttcacatgtatagattgcatttcttaggtgttttaattttttaaatatatttatgttttaaaaaaaaaaaaaaa

SEQ ID NO: 6 - DNA, 409 bp, Homo sapiens
catttgtcctgtttaattaggaaataaagacggtagagctagtcggaaaatgaatagttaatcgtggaggaggagaaaacatagctcagtactttgactagaggcagctgggaaagtgacgtcctgtagcatttgctgttctagaaagtacagagacacgtagtgaaaatgggaggatctagaaggaggctgtctcctgtgtagtgtatatttatctgtaagtgagccgttggggaaggattgaatacagagacgctgtctgcttgctgccttaagacagctagctgaattgctgattaacttttaaaatacccagcttggtttatttttcttagaatctgttgctaagactggggacgctgttttcttttacaaagggaaatctaagttaatttcaaggcattcgaaa

SEQ ID NO: 7 - DNA, 1088 bp, Homo sapiens
attacgattcttatgtgtgtgtgatatttaaagaaatgtgaaaatcccttttcacccttttcagtgtctagggagccagatttctttccgtctgttaatatataatacaatttctcacaaatatgaaagacccggtcttcaggttctctaaaataatttactgtgtcaagttttgataatattcctagctctctgaaaatgattgaatcaaataagtgtctatttttttttctgcaaacactacccgccagaacaatttccggttgttaaatagtaaagtcaccgttcctttggtaaggaatatttaaaggaactcccttggaaatgaattttgtaataggtgatttttattctgtttttactctatgtgatactgatccttgcactggatttcagtttggcaatatgctttttaaaactgggagtcttaaggtgactggatattagcatttttcactaagttttttggatttgaaagtaccttttgatattatttcttggtgaggctaaaaaaattgataaatagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaaaagatgagaaaaccatacaggagataaaagatgagttatatacatagaaaatgtctcataaatacctgaaatatgttatactttcaaaagcaggcatcaaaagggtatataaatgctatgatctaactttgttaacaaaaaattataaaactacagcaaataaccatgaagatttatagaaaacagaattagaagtaaatacagtaatatttaaca

SEQ ID NO: 8 - DNA, 350 bp, Homo sapiens
atagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaa

SEQ ID NO: 9 - DNA, 217 bp, Homo sapiens
aaagatgagaaaaccatacaggagataaaagatgagttatatacatagaaaatgtctcataaatacctgaaatatgttatactttcaaaagcaggcatcaaaagggtatataaatgctatgatctaactttgttaacaaaaaattataaaactacagcaaataaccatgaagatttatagaaaacagaattagaagtaaatacagtaatatttaaca

SEQ ID NO: 10 - DNA, 6 bp, Homo sapiens
tgggga

SEQ ID NO: 11 - DNA, 1896 bp, Homo sapiens
attacgattcttatgtgtgtgtgatatttaaagaaatgtgaaaatcccttttcacccttttcagtgtctagggagccagatttctttccgtctgttaatatataatacaatttctcacaaatatgaaagacccggtcttcaggttctctaaaataatttactgtgtcaagttttgataatattcctagctctctgaaaatgattgaatcaaataagtgtctatttttttttctgcaaacactacccgccagaacaatttccggttgttaaatagtaaagtcaccgttcctttggtaaggaatatttaaaggaactcccttggaaatgaattttgtaataggtgatttttattctgtttttactctatgtgatactgatccttgcactggatttcagtttggcaatatgctttttaaaactgggagtcttaaggtgactggatattagcatttttcactaagttttttggatttgaaagtaccttttgatattatttcttggtgaggctaaaaaaattgataaatagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaaaagatgagaaaaccatacaggagataaaagatgagttatatacatagaaaatgtctcataaatacctgaaatatgttatactttcaaaagcaggcatcaaaagggtatataaatgctatgatctaactttgttaacaaaaaattataaaactacagcaaataaccatgaagatttatagaaaacagaattagaagtaaatacagtaatatttaacaagggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaaccc

SEQ ID NO: 12 - DNA, 1680 bp, Homo sapiens
attacgattcttatgtgtgtgtgatatttaaagaaatgtgaaaatcccttttcacccttttcagtgtctagggagccagatttctttccgtctgttaatatataatacaatttctcacaaatatgaaagacccggtcttcaggttctctaaaataatttactgtgtcaagttttgataatattcctagctctctgaaaatgattgaatcaaataagtgtctatttttttttctgcaaacactacccgccagaacaatttccggttgttaaatagtaaagtcaccgttcctttggtaaggaatatttaaaggaactcccttggaaatgaattttgtaataggtgatttttattctgtttttactctatgtgatactgatccttgcactggatttcagtttggcaatatgctttttaaaactgggagtcttaaggtgactggatattagcatttttcactaagttttttggatttgaaagtaccttttgatattatttcttggtgaggctaaaaaaattgataaatagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaaagggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaaccc

SEQ ID NO: 13 - DNA, 1158 bp, Homo sapiens
atagctgacaaacctctgtaacctacaggtaggctagatcaatgttatacatttctaatgtcatggtgttgttaaagtagagaattttgaagagtagaaaatcgtagctgtcaaaatgcacaaagatgaacattcagaaagaattgctgaatcatcattgcttggcatatagagtaggcgttctcatttctttaaaggttaacacatgattattaccctttattatattctaaacatatatgtacattttcttttcctcataaaggttttctaacattttatttggactgtgtgcaattcttctgacttttcttatttatctctctaatagaaatgggaacatttttgaaagggattacctaaaatgatattatagggagatttttaaaaacttttttctgcgttttccaaaatctccaccaatgaatatatatttataattagggaaatgtgtttttgaaagaaaaaatatttctcatattactgccccttaagtggcatatccaaattttacattgatgcagttgcagttaagtcttgtaaggaacatgagtatgtactgtctgtaacctgatactttgttctagctttcctgcagcaccacagttctttaaatagggatcctgtggggcggctgcaggggacgggggttgaggggtggcagggacctcattcagttttagtttagttgaagtaactaagttcagatactaaaatttcaaaaataagtatttgtttgccagaactatgaactcatgtatactagaaataactagttagctaaaatcatgtttaagtcctttacttattttaaaataacgcatttgaattttattatagatactcccttaacaatgtgaaactagtacacaaactttctggcgtttctgattgatagaccccaggaattttgaagacgacgtaaattacttggaaatcctaatttaaacaacctgcgttttggtattatatacttaacagacaaatagcttaaaattaaaaaatccctaaagtagaattgtcgttccttttatctgtatattaactttaacttcttttaacatagggtgtttaatgactgtgatgattttcacctagtatggagtacataaatatatagttgtgacattctgtgttgaaatattggcatgttaaccc

SEQ ID NO: 14 - DNA, 5446 bp, Homo sapiens
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatccactagtaacggccgccagtgtgctggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 15 - DNA, 33 bp, Artificial Sequence
  Description of Artificial Sequence Synthetic Primer
ccgctcgagaattttgaagagtagaaaatcgta

SEQ ID NO: 16 - DNA, 34 bp, Artificial Sequence
  Description of Artificial Sequence Synthetic Primer
ccgctcgagggattacctaaaatgatattatagg

SEQ ID NO: 17 - DNA, 34 bp, Artificial Sequence
  Description of Artificial Sequence Synthetic Primer
ccgctcgagtagaattgtcgttccttttatctgt

SEQ ID NO: 18 - DNA, 34 bp, Artificial Sequence
  Description of Artificial Sequence Synthetic Primer
ccgctcgagattttctcctagtatggagtacata

SEQ ID NO: 19 - DNA, 33 bp, Artificial Sequence
  Description of Artificial Sequence Synthetic Primer
ccgctcgagcatttgtcctgtttaattaggaaa

SEQ ID NO: 20 - DNA, 27 bp, Artificial Sequence
  Description of Artificial Sequence Synthetic Primer
ccgctcgagggaaagtgacgtcctgta

SEQ ID NO: 21 - DNA, 28 bp, Artificial Sequence
  Description of Artificial Sequence Synthetic Primer
ccgctcgaggggaaggattgaatacaga

SEQ ID NO: 22 - DNA, 30 bp, Artificial Sequence
  Description of Artificial Sequence Synthetic Primer
cccaagcttttcgaatgccttgaaattaac

SEQ ID NO: 23 - DNA, 29 bp, Artificial Sequence
  Description of Artificial Sequence Synthetic Primer
cgacgcgtattacgattcttatgtgtgtg

SEQ ID NO: 24 - DNA, 24 bp, Artificial Sequence
  Description of Artificial Sequence Synthetic Primer
cgacgcgtagaacaatttccggtt

SEQ ID NO: 25 - DNA, 28 bp, Artificial Sequence
  Description of Artificial Sequence Synthetic Primer
cgacgcgtttgatattatttcttggtga

SEQ ID NO: 26 - DNA, 30 bp, Artificial Sequence
  Description of Artificial Sequence Synthetic Primer
cgacgcgtttcttatttatctctctaatag

SEQ ID NO: 27 - DNA, 28 bp, Artificial Sequence
  Description of Artificial Sequence Synthetic Primer
cgacgcgtcctctgtaacctacaggtag

SEQ ID NO: 28 - DNA, 29 bp, Artificial Sequence
  Description of Artificial Sequence Synthetic Primer
cgacgcgtatggtgttgttaaagtagaga

SEQ ID NO: 29 - DNA, 29 bp, Artificial Sequence
  Description of Artificial Sequence Synthetic Primer
cgacgcgtaaaatgcacaaagatgaacat

SEQ ID NO: 30 - DNA, 28 bp, Artificial Sequence
  Description of Artificial Sequence Synthetic Primer
cgacgcgttggcatatagagtaggcgtt

SEQ ID NO: 31 - DNA, 32 bp, Artificial Sequence
  Description of Artificial Sequence Synthetic Primer
cgacgcgtttaccctttattatattctaaaca

SEQ ID NO: 32 - DNA, 31 bp, Artificial Sequence
  Description of Artificial Sequence Synthetic Primer
cgacgcgtaaggttttctaacattttatttg

SEQ ID NO: 33 - DNA, 30 bp, Artificial Sequence
  Description of Artificial Sequence Synthetic Primer
ccgctcgagcctataatatcattttaggta

SEQ ID NO: 34 - DNA, 37 bp, Artificial Sequence
  Description of Artificial Sequence Synthetic Primer
ccgctcgagctattagagagataaataagaaaagtca

SEQ ID NO: 35 - DNA, 29 bp, Artificial Sequence
  Description of Artificial Sequence Synthetic Primer
ccgctcgagtcaccaagaaataatatcaa

SEQ ID NO: 36 - DNA, 25 bp, Artificial Sequence
  Description of Artificial Sequence Synthetic Primer
ccgctcgagaaccggaaattgttct

SEQ ID NO: 37 - DNA, 30 bp, Artificial Sequence
  Description of Artificial Sequence Synthetic Primer
ccgctcgagattacgattcttatgtgtgtg

SEQ ID NO: 38 - DNA, 34 bp, Artificial Sequence
  Description of Artificial Sequence Synthetic Primer
cccaagcttctcaattcccaaaatgcaataatag

SEQ ID NO: 39 - DNA, 24 bp, Artificial Sequence
  Description of Artificial Sequence Synthetic Primer
ccatccagtgttggtacattaatt

SEQ ID NO: 40 - DNA, 30 bp, Artificial Sequence
  Description of Artificial Sequence Synthetic Primer
cgcggatccatggggaaagactattattgc

SEQ ID NO: 41 - DNA, 29 bp, Artificial Sequence
  Description of Artificial Sequence Synthetic Primer
gctctagaattctatgaggcaggaagatg

SEQ ID NO: 42 - DNA, 24 bp, Artificial Sequence
  Description of Artificial Sequence Synthetic Primer
ccagcagacattgtttttatcatt

SEQ ID NO: 43 - DNA, 29 bp, Artificial Sequence
  Description of Artificial Sequence Synthetic Primer
attagtttacgagaggcattgtgtggctg

(19) United States
(12) Patent Application Publication
(10) Pub. No.: US 2006/0194235 A1
(43) Pub. Date: Aug. 31, 2006

(54) HLJ1 gene expression

(76) Inventors: Jian-Wei Chen, Fongyuan City (TW); Meng-Feng Tsai, Taipei City (TW); Chi-Chung Wang, Jhonghe City (TW); Pan-Chyr Yang, Taipei City (TW)

Correspondence Address: Finnegan, Henderson, Farabow,;Garrett & Dunner, L.L.P., 901 New York Avenue, N.W., Washington, DC, 20001, US

(21) Appl. No.: 11/360,671

(22) Filed: Feb. 24, 2006

Related U.S. Application Data
(60) Provisional application No. 60/655,877, filed on Feb. 25, 2005.

(51) Int. Cl. C12Q 1/68; G01N 33/574; C07H 21/04; C07K 16/30; C07K 14/82

(52) U.S. Cl. 435/6; 536/23.5; 435/7.23; 435/69.3; 435/320.1; 435/325; 530/350; 530/388.8; 514/12

(57) ABSTRACT

The human HLJ1 tumor suppressor gene is herein defined as regulated by promoter, enhancer, and silencer regions. HLJ1 promoter activity and gene expression are inversely correlated with metastatic ability. HLJ1 is highly expressed, and inducible, in cells with low metastatic ability and expressed to a lesser extent in highly metastatic cells. HLJ1 gene expression suppressed the growth of human lung adenocarcinoma cells in vitro, and inhibited tumor growth in vivo. It also impeded the motility of human adenocarcinoma cells and reduced the anchorage-independent growth capacity and invasiveness of metastatic lung adenocarcinoma cells. The degree to which human lung adenocarcinoma patients express HLJ1 predicts their survival prognosis and their probability of relapse.

### PRIORITY CLAIM

This application claims the benefit of U.S. Provisional 60/655,877, HLJ1 Gene Expression, filed in the U.S. Patent and Trademark Office Feb. 25, 2006, the disclosure of which is incorporated in its entirety.

### FIELD OF THE INVENTION

The invention relates to the 5′ regulatory region of the HLJ1 gene, including promoter and enhancer regions, and the use of these regions to regulate HLJ1 gene expression, thereby suppressing human lung adenoma cell growth and metastasis in vitro and in vivo.

### BACKGROUND ART

Heat Shock Proteins

Various stresses, for example, heat shock, heavy metals, ethanol, amino acid analogues, sodium arsenite, and oxidative stress, can induce a wide variety of organisms to synthesize heat shock proteins (HSPs) (1-3). HSPs have been classified into six major families by their molecular weights; these include Hsp100, Hsp90, Hsp70, Hsp60, Hsp40, and small heat shock proteins. HSPs can be targeted to different, specific, intracellular compartments (4). Within each family are constitutively expressed members and inducibly regulated members (4).

HSPs function as molecular chaperones to protect cells from environmental stress damage by binding to partially denatured proteins, dissociating protein aggregates, and promoting correct protein folding (5). They cooperate in transporting newly synthesized polypeptides to targeted organelles for packaging, degradation, or repair, and thus play a role in maintaining protein quality control (6). Members of the Hsp70 and the Hsp40 families work together as a chaperone system to minimize aggregation of newly synthesized proteins. A feature of Hsp40 chaperon proteins is an approximately 70-amino-acid-domain, the J domain, which orchestrates interactions with Hsp70 chaperones. Members of the Hsp40 family include Hsp40/DnaJ proteins, which have a conserved J domain (67). Hsp40/DnaJ proteins can act as cochaperones and specificity factors for Hsp70 family proteins (54). DnaJ has been reported to interact with DnaK to stimulate ATPase activity and to act as a chaperone in conjunction with DnaK (11).

Reports indicate that DnaJ-like proteins regulate cell mobility by regulating cytoskeleton formation. The DnaJ-like protein Mrj, which is in the Hsp40 family, was observed to bind directly to cytokeratin 18; microinjection of anti-Mrj antibody resulted in the disorganization of cytoskeletal filaments (61). Another DnaJ-like protein, ARG1, has also been reported to interact with the cytoskeleton (62).

DnaJ proteins have also been implicated in neoplastic transformation. For example, the SV40 large tumor antigen viral oncoprotein contains an amino-terminal J-domain which plays a role in SV40 transformation (64). Also, a human DnaJ protein homologue of the Drosophila Tid56 protein has been isolated using a yeast two-hybrid system with the human papilloma virus 16 viral oncoprotein E7 as bait (65). Loss of expression of the Drosophila DnaJ homologue hTid-1 correlates with a loss of differentiation capacity by neoplastic cells (66). Further, loss of expression of the Drosophila DnaJ homologue hTID 56 results in imaginal discs which fail to differentiate and form tumors (63).

Human Liver DnaJ-Like Protein

The human liver DnaJ-like protein is encoded by the human HLJ1 gene. It was first isolated from a human liver cDNA library by the yeast two-hybrid method using the S. pombe G protein β subunit as bait (7). DNA sequence analysis showed that it contained DnaJ and DnaG/F domain sequences like those of Hsp40 family members (7,8). Partial amino acid sequencing and cDNA cloning revealed that the human liver DnaJ-like protein is a mammalian homologue of a bacterial DnaJ heat shock protein (9, 10). To date, several other eukaryotic homologues of DnaJ-like Hsp40 proteins have been identified in various organisms ranging from yeast to human (12). The promoter elements of HLJ1 have not yet been characterized, and virtually nothing is currently known about the molecular mechanisms that regulate HLJ1 gene expression.

Metastasis

Metastasis, the migration of malignant cancer cells from their primary sites of origin to distant secondary sites within the body, requires altered expression of multiple genes in a multiple-step process. Cell adhesion, degradation of the surrounding extracellular matrix, migration, proliferation at a secondary site, and angiogenesis characterize the metastatic process (43, 44). Proteins including NM23, CD44, MTA1, MMPs, TIMPs, KAI1, E-cadherin, and KiSS1 have been reported to mediate metastasis (45-53); however, their molecular mechanisms are not clearly understood. Tumor cells obtain metastatic ability by coordinately expressing metastasis-promoting genes and down-regulating metastasis-suppressing genes. Therefore, correlating genes altered during the progression of a cancer with the metastatic phenotypes of cancer cells can lead to an understanding of how cells acquire metastatic phenotypes.

Selecting cells of increasingly invasive cancer cell populations from clonal cell lines has produced model cell lines with various, defined, metastatic abilities. The CL1 clonal cell line was derived from a human lung adenocarcinoma using a transwell invasion chamber assay (19, 24). These cell lines include, in increased order of metastatic ability, CL1-0 (lowest), CL1-1, CL1-5, and CL1-5-F4 (highest) (19, 24). Screening a panel of these lung cancer cell lines by cDNA microarray analysis identified dozens of metastasis-associated genes on a genome-wide scale (13), for example, CRMP-1, which had been characterized in the clinic as a metastasis mediator (35). Most of the genes identified by microarray analysis were involved in angiogenesis, cell motility, adhesion, or proliferation. This analysis tool identified HLJ1 as a gene with an expression profile that correlated with metastasis, but the functional relationship of HLJ1 with metastasis remains uncharacterized.

Lung Cancer

Lung cancer is among the cancers most likely to have undergone metastasis when it is detected. It is the most common cause of cancer death in the world, accounting for 12.3% of all cancer cases and 17.8% of all cancer deaths (36). In Taiwan, the mortality rate for lung cancer in 2002 was 41.12 and 19.38 per 100,000 among men and women, respectively (37). Lung cancers include small cell lung carcinomas (SCLC), which account for about 20% of lung cancers, and non-small cell lung carcinomas (NSCLC), which account for about 80% of lung cancers. Histological subtypes within NSCLCs include squamous cell carcinomas; large cell carcinomas; and adenocarcinomas, the most common histological subtype (38, 39).

Metastasis is an important parameter in determining lung cancer survival (36, 41, 42). Due to a lack of diagnostic tools for early detection and a lack of efficient treatment options effective against advanced disease, the overall five-year survival rate for lung cancer is less than 15% (4-6). When lung cancer is diagnosed and treated before it metastasizes, the five-year survival rate climbs to approximately 50-70%. However, once metastasis has occurred, the five-year survival rate drops to less than 5%.

### SUMMARY OF THE INVENTION

The present invention identifies the 5′ upstream regulatory region of the human HLJ1 tumor suppressor gene. The invention includes an isolated nucleic acid molecule comprising the 5′ regulatory region of the human HLJ1 gene, comprising nucleotides −2126 to +17 or one or more of its fragments or variants, and is contiguous with DNA encoding the human mRNA sequence designated NM_{—}007034 in the National Center for Biotechnology Information (NCBI) database (http://www.ncbi.nim.nih.gov/entrez/viewer.fcgi?db=nucleotide&val=24431959). The first nucleotide of the NM_{—}007034 mRNA corresponds to the nucleotide transcribed from the nucleotide designated +18 in FIG. 1. This invention identifies HLJ1 regulatory elements, characterizes their effects on cell proliferation and metastasis, and correlates HLJ1 regulation with tumor growth and with survival and recurrence rates of lung tumors in human cancer patients.

The invention provides an isolated nucleic acid molecule comprising the 5′ regulatory region of the human HLJ1 gene, comprising nucleotides −2126 to +17 of the human HLJ1 gene, or one or more fragments or variants thereof. This isolated nucleic acid molecule may be operably linked to a structural gene, for example a reporter gene, such as luciferase. The invention provides a vector comprising the nucleic acid molecule. It also provides a host cell transfected with the nucleic acid molecule. The host cell may be a cancer cell, for example, an adenocarcinoma cell. The host adenocarcinoma cell may comprise a cell line, including the cell lines CL1-0, CL1-1, CL1-5, CL1-5-F4, CL1-5/HLJ1, PCC1, PCY3-1, PCY4-2, and PCY4-5. The host adenocarcinoma cell may be a lung cell.

A nucleic acid molecule of the invention comprises a transcriptional start site located 176 base pairs (bp) upstream of a translational initiation site. Various embodiments of these nucleic acid molecules include one or more transcription factor YY1 binding sites at nucleotides −232 to −228, −211 to −207, −185 to −181; and −154 to −151. Various embodiments of the invention also comprise an enhancer region at nucleotides −2126 to −1039, a silencing element at nucleotides −1,255 to −1,039, and/or a GC box beginning at nucleotide −761. Various embodiments of the invention comprise a core promoter region at nucleotides −232 to +176.

The invention provides the HLJ1 gene with its 5′ flanking sequences which contain the regulatory promoter and enhancer sequences (SEQ. ID. NO.:1). The invention also provides the regulatory sequence itself within the 5′ flanking sequences (SEQ. ID. NO.:2). The invention further provides the HLJ1 gene linked to the promoter sequence (SEQ. ID. NO.:3) and the promoter sequence itself (SEQ. ID. NO.:4). The invention yet further provides the HLJ1 gene linked to the core promoter sequence (SEQ. ID. NO.:5) and the core promoter sequence itself (SEQ. ID. NO.:6). The invention provides the enhancer sequence (SEQ. ID. NO.:7) and the minimal enhancer sequence (SEQ. ID. NO.:8) of the HLJ1 gene.

The invention provides a silencing element (SEQ. ID. NO.:9) in the 5′ flanking region of the HLJ1 gene, the deletion of which can lead to increased transcription of the HLJ1 gene and thereby slow metastasis. The invention also provides the transcription start site (SEQ. ID. NO.:10) of the HLJ1 gene.

The invention provides the enhancer sequence linked to the core promoter sequence (SEQ. ID. NO.:11) and provides a partial enhancer sequence linked to the core promoter sequence (SEQ. ID. NO.:12), the latter of which can produce HLJ1 transcripts in a reporter gene assay. The invention also provides the minimal enhancer sequence linked to the minimal promoter sequence (SEQ. ID. NO.:13). The invention further provides fragments and variants of SEQ. ID. NOS.:1-13.

The invention provides the transcription factor YY1 (SEQ. ID. NO.:14), the over-expression of which can lead to increased HLJ1 transcription. The invention also provides various novel forward and reverse PCR primers designed to amplify various fragments of the flanking region of the HLJ1 gene (SEQ. ID. NO.:15-SEQ. ID. NO.:22), designed to amplify the HLJ1 enhancer (SEQ. ID. NO.:23-SEQ. ID. NO.:36), and designed from the 5′ end of the known HLJ1 cDNA sequence to clone and sequence the 5′ flanking region of the HLJ1 gene (SEQ. ID. NO.:37 and SEQ. ID. NO.:38). The invention provides a PCR primer (SEQ. ID. NO.:39) designed to amplify cDNA obtained from reverse transcription of RNA isolated from CL1-0 cells and used to identify the transcription start site by 5′-rapid amplification of cDNA ends (5′-RACE). The invention also provides novel forward and reverse PCR primers designed to amplify the HLJ1 coding region (SEQ. ID. NO.:40 and SEQ. ID. NO.:41 respectively). The invention further provides novel forward and reverse primers for the RT-PCR analysis of HLJ1 gene expression (SEQ. ID. NO.:39 and SEQ. ID. NO.:40, respectively). The invention yet further provides a novel probe sequence (SEQ. ID. NO.:43) to detect and quantify the RT-PCR product.

The invention provides a molecule comprising the 5′ regulatory region of the human HLJ1 gene, or variants or fragments thereof, operably linked to a structural gene, e.g., a reporter gene, such as luciferase. The invention provides vectors comprising the 5′ regulatory region of the human HLJ1 gene or fragments thereof, and host cells transfected with the 5′ regulatory region of the human HLJ1 gene or fragments thereof. The host cell can be a cancer cell, e.g., an adenocarcinoma cell. The host cell can belong to a cell line, e.g., CL1-0, CL1-1, CL1-5, CL1-5-F4, such as a lung adenocarcinoma cell, and the adenocarcinoma cell can be of human origin.

The invention provides an isolated nucleic acid comprising at least 2300, 2400, 3000, 3300, 3500, 3800, 4000, or 4300 consecutive nucleotides of the complement of SEQ. ID. NO.:1. The invention provides an isolated nucleic acid molecule comprising at least 200, 500, 1000, 1500, or 2000 consecutive nucleotides of the complement of SEQ. ID. NO.:2. The invention provides an isolated nucleic acid molecule comprising at least 2300, 2400, 3000, or 3300 consecutive nucleotides of the complement of SEQ. ID. NO.:3. The invention provides an isolated nucleic acid molecule comprising at least 200, 400, 600, or 800 consecutive nucleotides of the complement of SEQ. ID. NO.:4. The invention provides an isolated nucleic acid molecule comprising at least 2300 or 2400 consecutive nucleotides of the complement of SEQ. ID. NO.:5. The invention provides an isolated nucleic acid molecule comprising at least 250, 350, or 400 consecutive nucleotides of the complement of SEQ. ID. NO.:6. The invention provides an isolated nucleic acid molecule comprising at least 600, 800, or 1000 consecutive nucleotides of the complement of SEQ. ID. NO.:7. The invention provides an isolated nucleic acid molecule comprising at least 200, 300, or 330 consecutive nucleotides of the complement of SEQ. ID. NO.:8. The invention provides an isolated nucleic acid molecule comprising at least 200 consecutive nucleotides of the complement of SEQ. ID. NO.:9. The invention provides an isolated nucleic acid molecule comprising at least five consecutive nucleotides of the complement of SEQ. ID. NO.:10. The invention provides an isolated nucleic acid molecule comprising at least 200, 500, 800, 1000, 1500, 1700, or 1800 consecutive nucleotides of the complement of SEQ. ID. NO.:11. The invention provides an isolated nucleic acid molecule comprising at least 200, 500, 800, 1000, 1600, or 1700 consecutive nucleotides of the complement of SEQ. ID. NO.:12. The invention provides an isolated nucleic acid molecule comprising at least 200, 500, 800, 1000, or 1100 consecutive nucleotides of the complement of SEQ. ID. NO.:13. The invention also provides a single stranded oligonucleotide comprising a sequence selected from SEQ. ID. NO.:1, SEQ. ID. NO.:2, SEQ. ID. NO.:3, SEQ. ID. NO.:4, SEQ. ID. NO.:5, SEQ. ID. NO.:6, SEQ. ID. NO.:7, SEQ. ID. NO.:8, SEQ. ID. NO.:9, SEQ. ID. NO.:10, SEQ. ID. NO.:11, SEQ. ID. NO.:12, and SEQ. ID. NO.:13.

In another aspect, the invention provides a method of identifying a compound that modulates HLJ1 gene expression by providing a cell transiently or stably transfected with an isolated nucleic acid molecule comprising one or more of SEQ. ID. NOS.:1-13, or a fragment or variant of SEQ. ID. NOS.:1-13, contacting the cell with a test compound, and determining the level of expression of the enhanced green fluorescent protein (EGFP) gene in the presence of the test compound, wherein a low level of expression of EGFP is an indication that the test compound inhibits the promoter activity of the HLJ1 gene.

In a further aspect, the invention provides a method of screening for modulators of HLJ1 gene expression by providing a cell transiently or stably transfected with HLJ1 DNA comprising one or more of SEQ. ID. NOS.:1-13, or a fragment or variant of SEQ. ID. NOS.:1-13, contacting the cell with a candidate modulator, and determining the ability of the candidate modulator to affect one or more of the growth, metastatic, and/or invasive properties of the cell. The invention also provides a method of diagnosing cancer by determining the level of HLJ1 gene expression in a mammalian tissue sample in comparison to non-malignant control tissue, wherein the level of HLJ1 expression correlates with the presence of cancer. This method can be used to diagnose lung cancer, for example, lung adenocarcinoma.

In yet another aspect, the invention provides a method of determining the metastatic ability of a cell by providing a cell of unknown metastatic ability, determining its level of HLJ1 gene expression, and comparing the HLJ1 expression level of the cell with unknown metastatic ability to a positive control of known metastatic ability and to a non-metastatic negative control cell, wherein the level of HLJ1 expression negatively correlates with the metastatic ability of the cell. The invention also provides a method of decreasing the metastatic ability of a cell possessing such ability by increasing its HLJ1 gene expression, wherein the expression of the HLJ1 gene negatively correlates with the metastatic ability of the cell. The invention further provides a method of decreasing the metastatic ability of a cell possessing such ability by transfecting the cell with a nucleic acid molecule corresponding to the human HLJ1 gene or a fragment thereof, and expressing the nucleic acid molecule within the cell, wherein the expression of the nucleic acid molecule negatively correlates with the metastatic ability of the cell. This method can be practiced on a cancer cell, e.g., a human lung adenocarcinoma cell.

The invention provides a method of inhibiting cell proliferation by increasing its HLJ1 gene expression, wherein the expression of the HLJ1 gene correlates with an inhibition of cell proliferation. The invention also provides a method of inhibiting cell proliferation by transfecting a cell with a nucleic acid molecule corresponding to the human HLJ1 gene or a regulatory fragment or variant thereof, and expressing the nucleic acid molecule within the cell, wherein the expression of the HLJ1 gene correlates with an inhibition of cell proliferation. This method can be practiced on a cancer cell, e.g., a human lung adenocarcinoma cell. The invention further provides a method of decreasing the invasive ability of a cell possessing such ability by transfecting the cell with a nucleic acid molecule corresponding to the human HLJ1 gene or a regulatory fragment or variant thereof, and expressing the nucleic acid molecule within the cell, wherein the expression of the nucleic acid molecule correlates with decreased invasive ability. This method can also be practiced on a cancer cell, e.g., a human lung adenocarcinoma cell.

The invention also provides a method of diagnosing the presence of cancer by providing a mammalian tissue sample and determining the level of HLJ1 gene expression in comparison to non-malignant control tissue, wherein the level of expression negatively correlates with the presence of cancer, for example, lung adenocarcinoma.

The invention further provides methods of predicting the quantitative probability of surviving lung cancer and of the recurrence of lung cancer in a patient diagnosed with lung cancer by measuring the expression of HLJ1 mRNA in the patient's cancer cells and applying a statistical method of analysis, e.g., the Kaplan Meier method, wherein the statistical method predicts the probability of survival or recurrence.

In another aspect, the invention provides a therapeutic composition comprising a modulator of HLJ1 and a pharmaceutically acceptable carrier. This composition can be used to treat a patient in need of such treatment and can be administered in any manner, including orally, parenterally, by implantation, by inhalation, mucosally, intranasally, intravenously, intra-arterially, intracardiacally, subcutaneously, intradermally, intraperitoneally, transdermally, intraventricularly, intracranially, and/or intrathecally, wherein administering the composition treats the patient.

The invention further provides a method of treating a patient with lung adenocarcinoma by transfecting one or more the patient's adenocarcinoma cells with a nucleic acid molecule that increases the promoter activity of HLJ1, thereby increasing HLJ1 expression in one or more of the patient's adenocarcinoma cells.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention as claimed. The accompanying drawings, which are incorporated in, and constitute a part of, this specification illustrate several embodiments of the invention and, together with the description, serve to explain the principles of the invention.

### BRIEF DESCRIPTION OF THE DRAWINGS

FIG. 1. Nucleotide Sequence of the 5′ Flanking Region of the HLJ1 Gene

The nucleotide sequence of the 5′ flanking region of the human HLJ1 gene sequence is numbered relative to the mRNA transcription initiation site, which is indicated by a horizontal arrow and designated as +1. Transcription factor binding site consensus sequences are underlined and the corresponding transcription factors are shown below the underlines. The first codon (ATG) is denoted in bold, and the beginning of the coding region is denoted with italic print, with alternate codons underlined. The first twelve encoded amino acids are shown below the nucleotide sequence.

FIG. 2. Reporter Assay for the 5′ Upstream Region of Human HLJ1

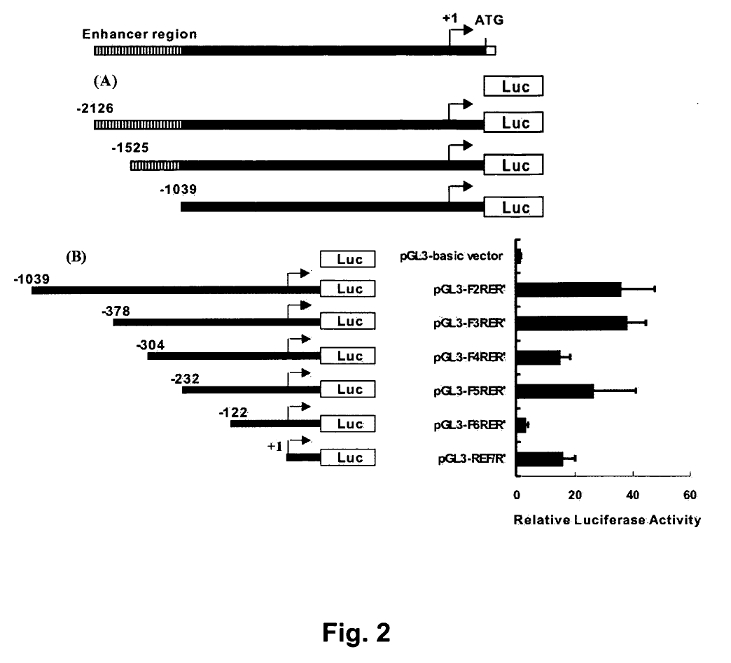

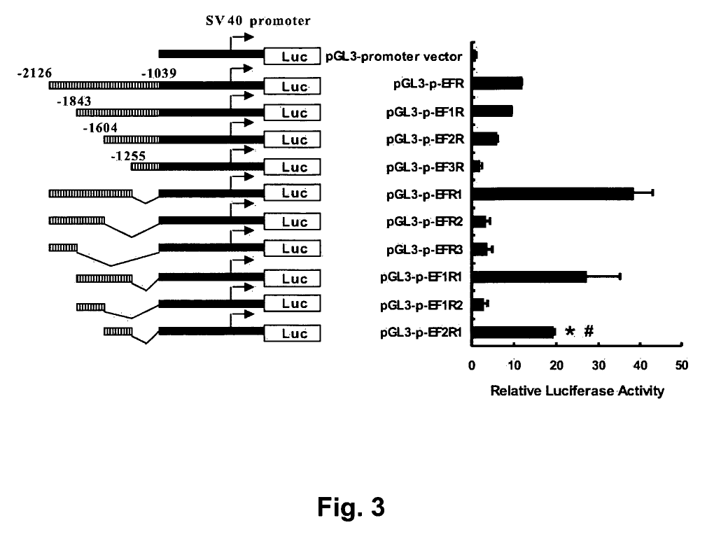

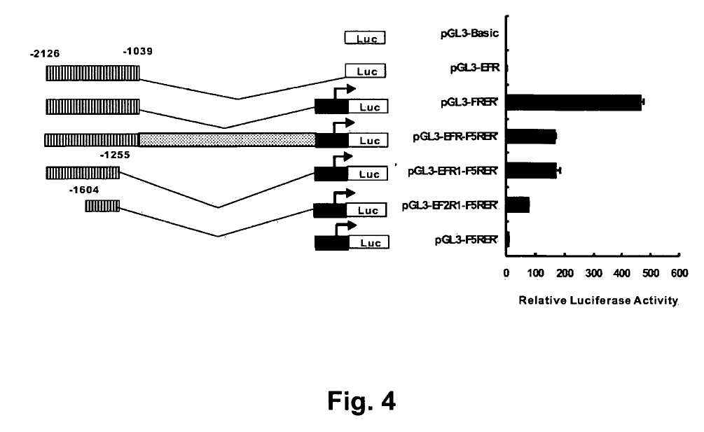

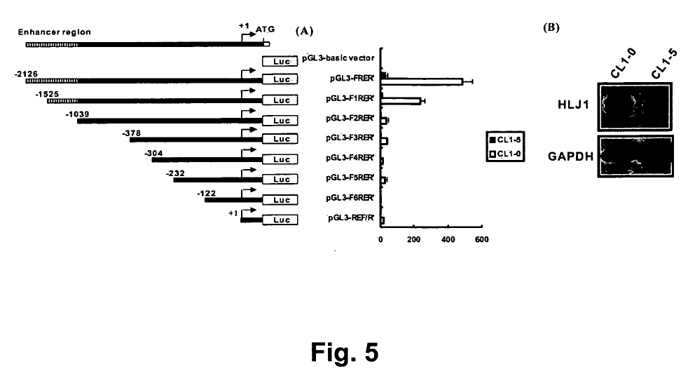

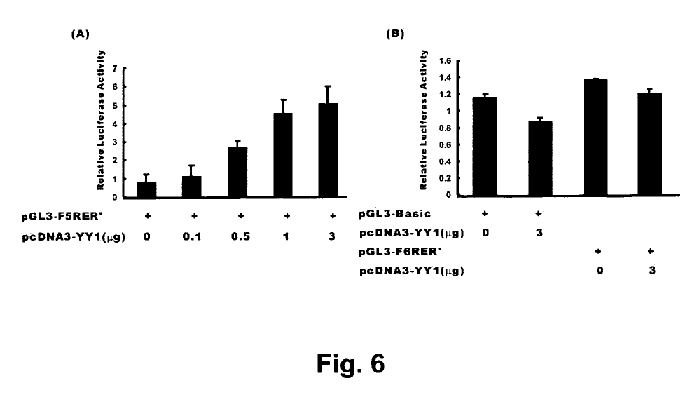

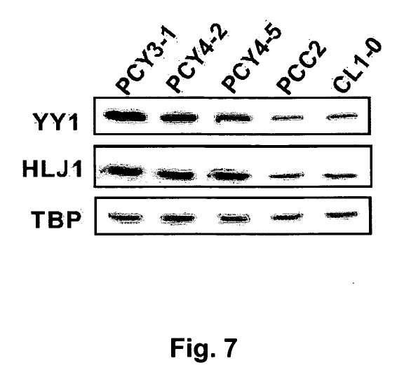

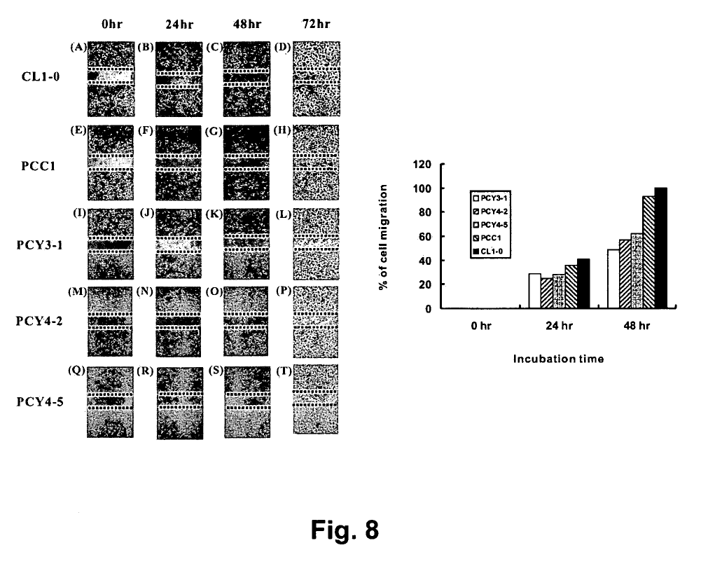

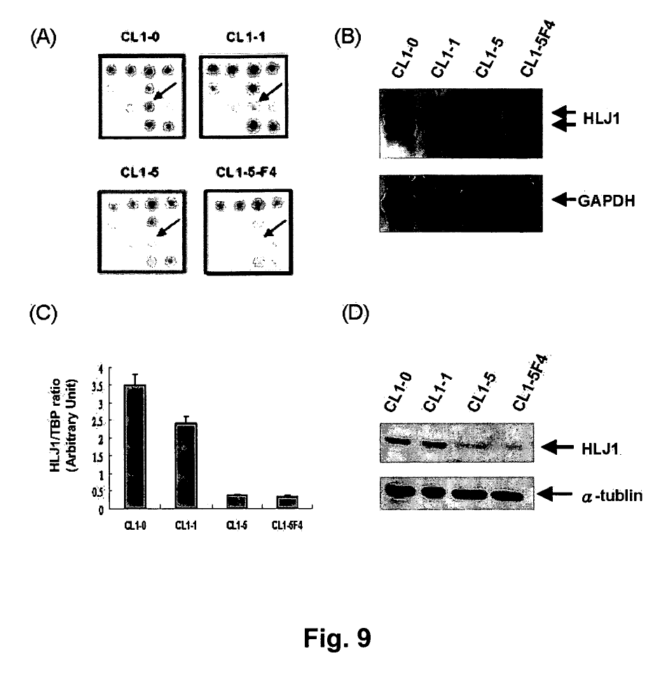

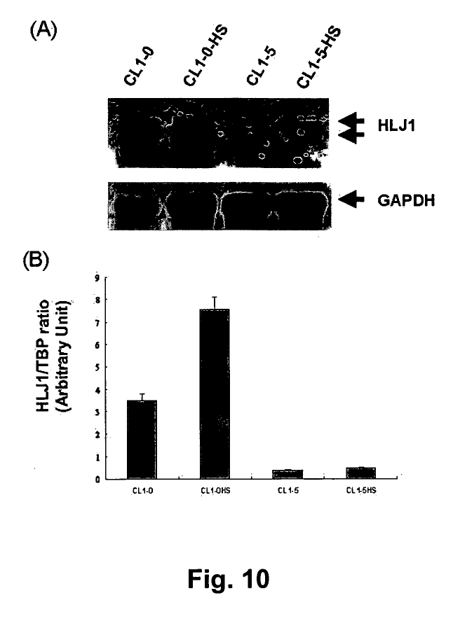

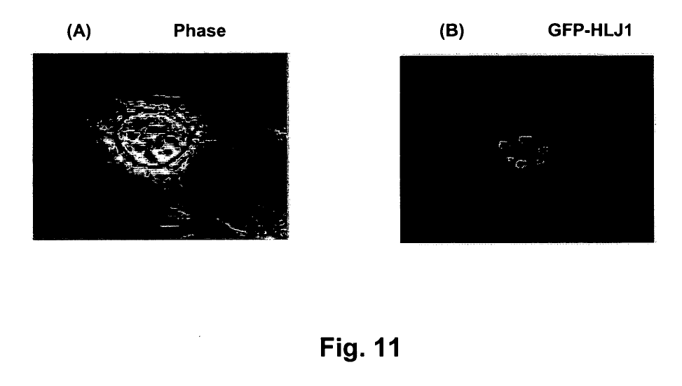

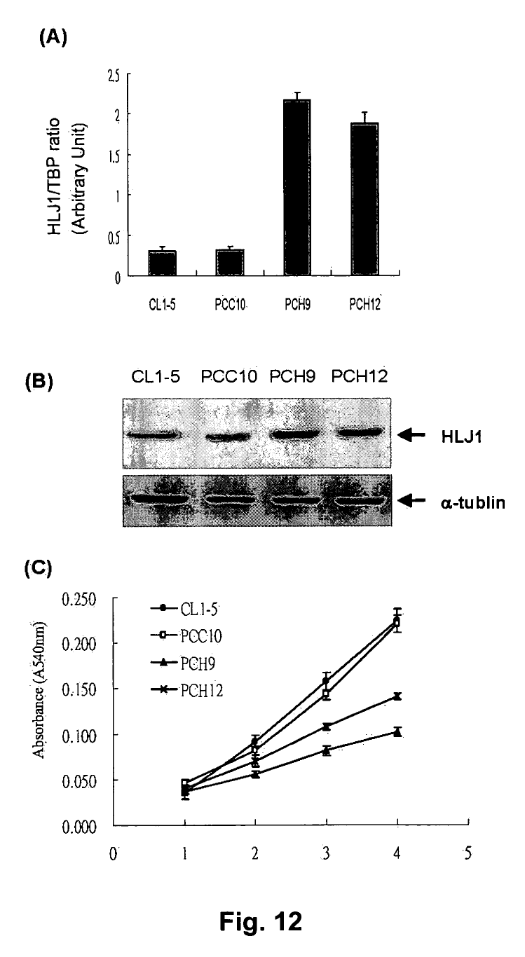

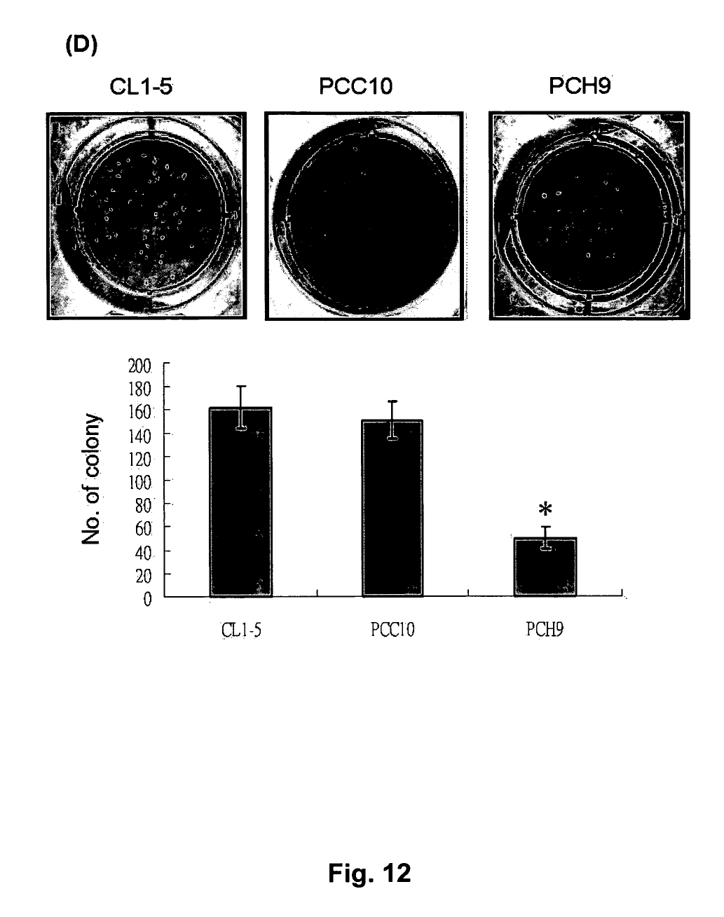

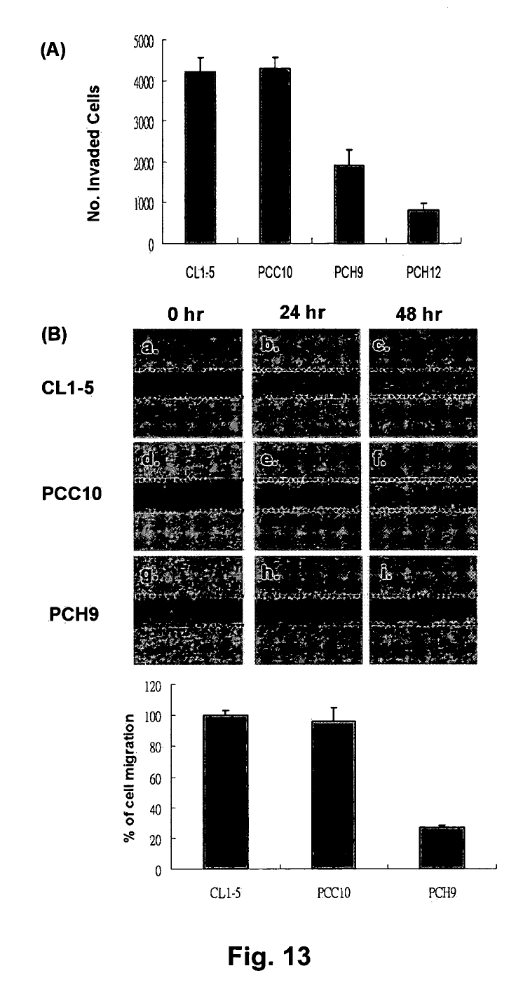

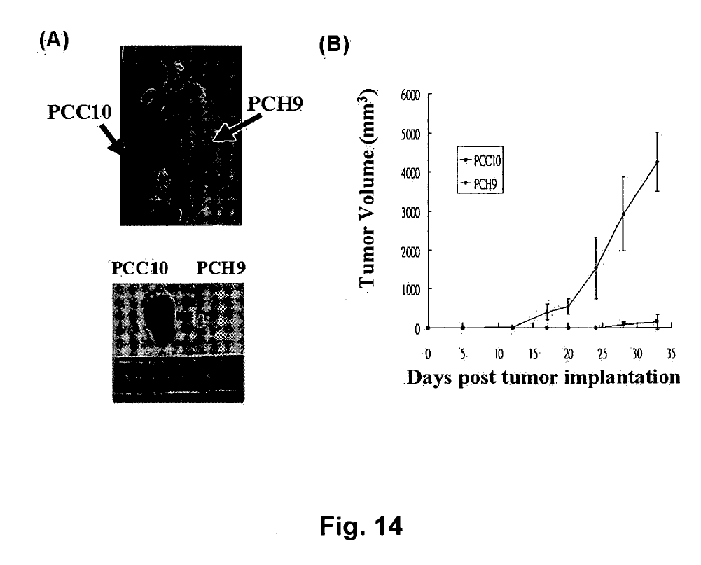

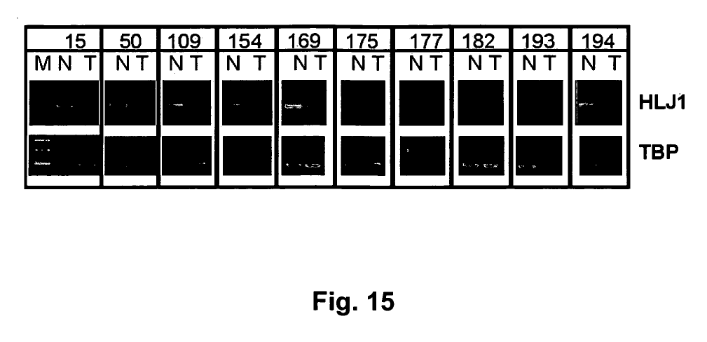

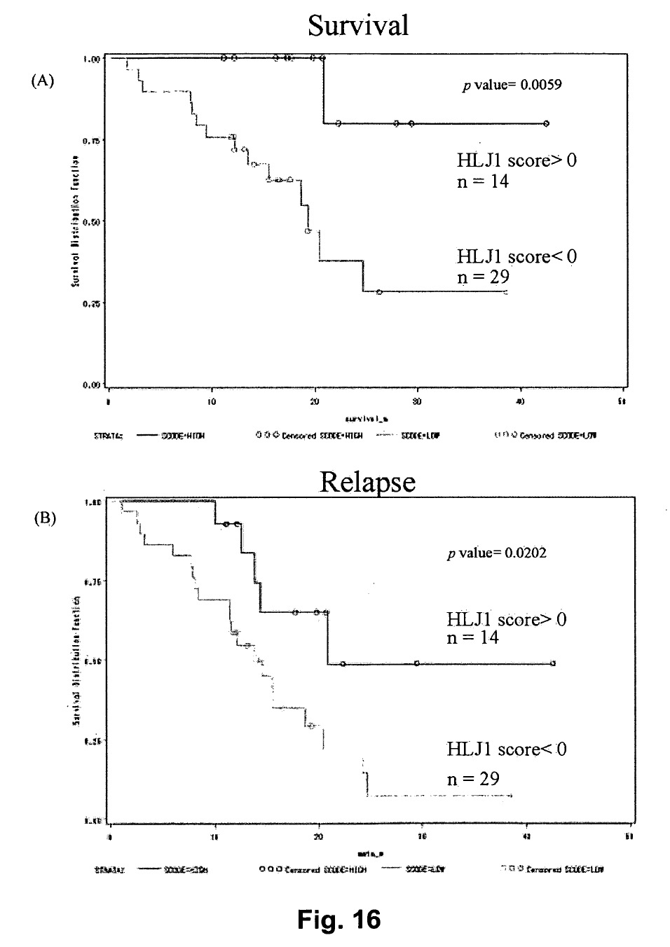

The 5′ flanking region and part of the coding region of the human HLJ1 gene are shown in diagrammatic form at the top of FIG. 2. The enhancer region is hatched, the mRNA initiation site is indicated by a horizontal arrow and designated as +1, and the first codon is labeled ATG.

(A) FIG. 2A shows constructs with the entire enhancer region, with part of the enhancer region, and missing the enhancer region.

(B) FIG. 2B shows constructs with the entire promoter region, with various parts of the promoter region, and missing the promoter region. Construct names are provided on the right side of FIG. 2B, along with a measure of promoter activity. The pGL3 basic vector has no promoter. The names are defined by the nucleotide designated as the 5′ nucleotide, as further described below. For example, pGL3-F2RER′ is a pGL3 vector with a fragment of the 5′ flanking region beginning at nucleotide −1039, and ending with the 3′ luciferase reporter gene, corresponding to the HLJP-RER promoter reverse primer, as shown in Table 1. Promoter activity is expressed as relative luciferase activity.

FIG. 3. Deletion Mapping of the Human HLJ1 Enhancer Region

HLJ1 enhancer deletion mutants were subcloned into an enhancer-less pGL3-SV40-promoter vector, upstream of the luciferase gene. The numbers on the left of each enhancer deletion construct shown in the left panel refer to the beginning position of the promoter fragments. The enhancer regions are shown as hatched bars, the SV40 promoter region of the enhancer-less vector pGL3-p-EFR is shown as a black bar, the mRNA initiation site is depicted with an arrow, and the promoter activity, expressed as relative luciferase activity, is shown to the right of each construct.

FIG. 4. HLJ1 Enhancer-Basal Promoter Recombinant Constructs

CL1-0 cells were transiently transfected with each of the HLJ1 enhancer-basal promoter recombinant constructs shown in the left panel. The promoter activity, expressed as luciferase activity, was normalized to the pGL3-basic vector and shown in the right panel.

FIG. 5. Comparison of Promoter Activity in CL1-0 and CL1-5 Cells

(A) CL1-0 and CL1-5 cells were transiently transfected with different HL1J reporter constructs and the promoter activity was measured 48 hr after transfection. The promoter activity of each construct was normalized to the activity of co-transfected pSV-β-Gal (mean±SEM). Promoter activity in CL1-5 cells is shown with black bars and promoter activity in CL1-0 cells is shown with white bars.

(B) Northern blot analysis of HLJ1 mRNA in CL1-0 and CL1-5 cells was performed as described in Example 5. The glyceraldehyde 3-phosphate dehydrogenase (GAPDH) probe is an internal control for the quantity of mRNA.

FIG. 6. Overexpression of YY1 Stimulates HLJ1 Promoter Activity

(A) CL1-0 cells were co-transfected with 1.5 μg pGL3-F5RER′ and varying amounts of the plasmid pcDNA3-YY1, cultured for 44 hours, and assayed for promoter activity, as described in Examples 4 and 6. Data represent the mean±S.D. from three independent experiments.

(B) The pGL3-basic vector plasmid (negative control) and the pGL3-F6RER′ plasmid constructs were co-transfected with either 0 or 31 g pcDNA3-YY1 plasmid, and assayed as described in (A).

FIG. 7. Western Blot Analysis of HLJ1 Expression

The expression of HLJ1 protein was compared in cell lines transfected with the transcription factor YY1. Equal amounts of a nuclear protein extract of each of the cell clones was analyzed by Western blot analysis, as described in Example 7. In the top panel, the cell extracts were probed with YY1-specific monoclonal antibody, then re-probed with HLJ1 polyclonal antibody (middle panel), and TBP monoclonal antibody (lower panel) as an internal control. The HLJ1 polyclonal antibody was made as described in Example 8. The cell lines examined include the transfected cell lines PCY3-1, PCY4-2, and PCY4-5, mock-transfected CL1-0 cells (PCC2) and untransfected CL1-0 cells.

FIG. 8. HLJ1 Expression Inhibits CL1-0 Cell Migration

Equivalent numbers of confluent CL1-0, PCC1, PCY3-1, PCY4-2, and PCY4-5 cells were wounded as described in Example 9 and photographed 0, 24, 48, and 72 hours after wounding. Panels (A), (E), (I), (M), and (O) show the appearance of the wound margin immediately upon scraping and washing. Panels (B), (F), (J), (N), and (R) show the appearance of the wound margin 24 hours after wounding. Panels (C), (G), (K), (O), and (S) show the appearance of the wound margin 48 hours after wounding. Panels (D), (H), (L), (P), and (T) show the appearance of the wound margin 72 hours after wounding. All panels were stained by crystal violet to assay for the presence of viable cells in the wounded region. The bar graph in the right panel shows the percentage of cells migrating into the wound in comparison to untransfected cells. Migration was highest in the untransfected and mock-transfected cells, and was reduced in cells transfected with YY1.

FIG. 9. Differential Expression of HLJ1 mRNA in Lung Cancer Cell Lines

(A) A DNA microarray screen of four lung cancer cell lines, as described in Example 10, demonstrated that HLJ1 expression correlated inversely with the metastatic ability of the cell line (CL1-0<CL1-1<CL1-5<CL1-5F4). The arrow points to the microarray address of the HLJ1 gene. Colorimetric detection demonstrated that HLJ1 gene expression is greatest in CL1-0 cells and progressively diminished in CL1-1 and CL1-5. The lowest level of HLJ1 expression was observed in the most highly metastatic cells CL1-5-F4.

(B) Northern blot analysis of HLJ1 mRNA transcribed from full-length HLJ1 cDNA was performed as described in Example 5. Messenger RNA levels inversely correlated with the metastatic ability of the cell line. The GADPH probe is an internal control for the quantity of mRNA.

(C) RTQ RT-PCR was performed as described in Example 11, and also demonstrated that mRNA levels were inversely correlated with the metastatic ability of the cell line. The HLJ1 mRNA levels were expressed in relation to the internal control TBP mRNA.

(D) Western blot analysis was performed as described in Example 7, and demonstrated that the expression of HLJ1 protein was higher in cells with low metastatic ability (CL1-0 and CL-1) than in cells with high metastatic ability (CL1-5 and CL1-5F4). The α-tubulin probe is an internal control for the quantity of mRNA.

FIG. 10. Effect of Heat Shock on HLJ1 Expression in CL1-0 and CL1-5 Cells

(A) Northern blot analysis of HLJ1 mRNA demonstrated that a heat shock (HS) treatment of 45° C. for 30 min induced HLJ1 mRNA expression in CL1-0 cells, but not in the more highly metastatic CL1-5 cells. The GADPH probe is an internal control for the quantity of mRNA.

(B) RTQ RT-PCR also demonstrated that HS can induce HLJ1 mRNA expression in CL1-0 cells, but not in CL1-5 cells. CL1-0 cells expressed more than twice as much HLJ1 mRNA following HS (CL1-0HS) than before HS. Heat shock did not induce HLJ1 mRNA in CL1-5 cells, which expressed HLJ1 mRNA at a lower level than CL1-0 cells in both the absence (CL1-5) and the presence (CL1-5HS) of HS.

FIG. 11. Subcellular Localization of EGFP-HLJ1 in CL1-5 Cells

(A) The phase contrast micrograph shows a CL1-5 cell transfected with pEGFP-HLJ1, a mammalian transfection vector comprising the full coding region of HLJ1 cDNA fused to an enhanced green fluorescent protein (EGFP) gene.

(B) The scanning confocal micrograph shows a CL-1 cell transfected with pEGFP-HLJ1 and examined by indirect immunofluorescence, as described in Example 12. HLJ1 was localized to the cell nucleus, particularly the nucleoli.

FIG. 12. HLJ1 Suppressed Human Lung Adenoma Cell Growth In Vitro

(A) CL1-5 cells were stably transfected with the full-length HLJ1 pCDNA3-HLJ1 plasmid as described in Example 4. Single colonies were isolated and the levels of HLJ1 mRNA were measured by RTQ RT-PCR, as described in Example 11. CL1-5 cells were compared to transfection control cells (PCC10) and two colonies, namely PCH9 and PCH12 were transfected with HLJ1 pCDNA3-HLJ1. The HLJ1 mRNA levels were expressed in relation to the internal control TBP mRNA. PCH9 and PCH12 were transfected with HLJ1 pCDNA3-HLJ1. The HLJ1 mRNA levels were expressed in relation to the internal control TBP mRNA.

(B) The levels of HLJ1 protein in CL1-5 cells, the transfection control cells (PCC10), and the transfected PCH9 and PCH12 cells are shown by Western blot. The α-tubulin probe is an internal control for the quantity of mRNA.

(C) The proliferation rate of CL1-5 cells, PCC10 cells, PCH9 cells, and PCH12 cells was compared as described in Example 13. Transfected PCH9 and PCH12 cells proliferated more slowly than the untransfected CL1-5 and PCC10 cells.

(D) Anchorage-independent growth was compared in CL1-5, PCC10, and PCH9 cells as described in Example 14. After 14 days in soft agar culture, the colonies were stained with crystal violet. The number of colonies larger than 1 mm were counted and the results shown by the bar graph, expressed as the mean+/−standard deviation of triplicate samples. The * denotes p<0.005 v. controls, Student's t-test. Colony formation was assessed in three independent experiments.

FIG. 13. HLJ1 Reduced Invasion and Migration of CL1-5 Cells In Vitro

(A) The invasive potential of CL1-5/HLJ1 transfected cells was determined using a Matrigel™ invasion assay as described in Example 15.

(B) The migratory ability of CL1-5/HLJ1 transfected cells was determined using the cell migration assay described in Example 9. The appearance of the wound margin of confluent CL1-5 cells, PCC10 mock transfectants, and PCH9 HLJ1 transfectants is shown immediately after wounding (a, d, g), 24 hr after wounding (b, e, h), and 48 hr after wounding (c, f, i). Results are representative of three similar experiments from independently transfected groups of CL1-5 cells. The percentage of

FIG. 14. HLJ Inhibited In Vivo Tumor Growth

(A) Tumor development in SCID mice was examined as described in Example 16 following injection of 5×10⁶ untransfected PCC10 or transfected PCH9 cells. The appearance of in vivo tumors is shown in the top panel, and the relative size of the excised tumor is shown in the bottom panel.

(B) The in vivo volume of the tumors formed in the mice injected with PCC10 cells was compared to the volume of tumors formed by the PCH9 cells in the days following tumor implantation.

FIG. 15. Expression of HLJ1 mRNA in Human Lung Cancer

Ten human lung cancer tissue specimens were compared to adjacent normal lung tissue using semiquantitative RT-PCR. The expression of the HLJ1 product resulting from the amplification of the QHLJ1 forward and QHLJ1 reverse primers, as described in Example 2, was compared in DNA prepared from the tumor specimens (T), and the normal adjacent tissue (N). Molecular weight standards (100 bp ladder) are shown in lane M. The TBP probe is an internal control for the quantity of mRNA.

FIG. 16. Kaplan-Meier Survival and Relapse Plots

Kaplan-Meier plots for patients with lung adenocarcinoma show that survival rates were greater and recurrence rates were lesser in patients that expressed a higher than average amount, compared to patients that expressed a lower than average amount of HLJ1 mRNA. “Average” was calculated as the mean HLJ1 mRNA, standardized to control TATA box-binding mRNA, of all patients examined. Patients were designated as “high expressers” if they expressed a level of HLJ1 mRNA above the mean (HLJ1 score>0), and as “low expressers” if they expressed a level of HLJ1 were designated as “high expressers” if they expressed a level of HLJ1 mRNA above the mean (HLJ1 score>0), and as “low expressers” if they expressed a level of HLJ1 mRNA below the mean (HLJ1 score<0). P values were obtained using the log rank test.

(A) Kaplan-Meier survival plots for patients with lung adenocarcinoma show that survival rates were higher for “low expressers” than for “high expressers” (p=0.0202).

(B) Kaplan-Meier relapse plots for patients with lung adenocarcinoma show that relapse rates were higher for “low expressers” than for “high expressers” (p=0.0059).

### DETAILED DESCRIPTION OF THE INVENTION

Definitions

A “nucleic acid molecule” is a nucleotide polymer of any length. It can comprise deoxyribonucleotides, ribonucleotides, and/or their analogs. Nucleic acid molecules can be naturally occurring or synthetic analog, as known in the art.

An “isolated nucleic acid molecule” is a nucleic acid molecule, including a synthetic analog, that is separated from other nucleic acid molecules present in the natural source of the nucleic acid. An “isolated” nucleic acid molecule, such as a cDNA molecule, can be substantially free of other cellular material or culture medium when produced by recombinant techniques, or substantially free of chemical precursors or other chemicals when chemically synthesized.

A “variant” refers to molecule that differs from a referent molecule. Variants may be present in the normal physiological state, e.g., variant alleles such as SNPs and splice variants or may be present in pathological states, such as disease-degenerate variants, the latter of which have a different codon encoding the same amino acid or a different amino acid that carries out the same function.

A “complement” is a nucleotide sequence that is complementary to another sequence through base pairings.

A “gene” is an open reading frame encoding one or more specific RNA and/or polypeptide. It can include coding sequences, noncoding regulatory sequences, and/or introns.

A “transcriptional start site” is the first DNA nucleotide that is transcribed into RNA. This nucleotide is designated +1.

A “translation initiation site” is the first RNA nucleotide that is translated into a polypeptide. Translation initiation sites usually comprise the codon ATG.

A “promoter” is a region of DNA that binds RNA polymerase before initiating the transcription of DNA into RNA. The promoter directs the RNA polymerase to bind to DNA, to open the DNA helix, and to begin RNA synthesis.

A “core promoter” is the minimal promoter sequence required for sustained levels of promoter activity, such that removal of additional nucleotides decreases the activity.

An “enhancer” is a region of DNA that can increase the transcription of the gene into mRNA. Enhancers alone are not sufficient to cause the gene to be expressed.

A “silencing element” is a region of DNA that decreases transcription.

A “GC box” is a transcription factor binding site within a promoter region of mammalian cells with the general consensus sequence of GGGCGG.

A “transcription factor” is an endogenous substance, usually a protein, which acts to initiate, stimulate, or terminate the production of RNA from DNA. Transcription factors may bind to specific stimulatory sequences, such as promoters. They may activate transcription by RNA polymerases.

A “vector” is an agent, e.g., a virus or a plasmid, used to transmit genetic material to a cell or other organism.

A “host cell” is an individual cell, cell line, cell culture, or cell in vivo, which has been or can be a recipient of polynucleotides or polypeptides, for example, a recombinant vector, an isolated polynucleotide, or a fusion protein. Host cells include progeny of a single host cell. A host cell includes cells transformed, transfected, transduced, or infected in vivo or in vitro.

A “cell line” is a population of cells that is capable of proliferating indefinitely in culture.

“Transfection” and “transformation” are two methods for introducing DNA into a recipient eukaryotic cell and its subsequent integration into the recipient cell's chromosomal DNA.

“Operably linked” refers to nucleotide sequences that are associated or connected in such a manner that their transcription or translation can be associated or connected, e.g., they can be transcribed or translated together.

“Physiological conditions” refer to conditions found in vivo, or, alternatively, to in vitro reaction conditions intended to mimic or approximate those found in vivo. With regard to in vitro conditions which seek to approximate in vivo conditions, consideration should be given to pH, salt concentrations, buffering capacity, temperature, and such other parameters as may be deeded necessary in the particular circumstance. As those in the art will appreciate, what constitutes physiological conditions in a given situation may depend on many factors, such as the type of organism being considered, the environment inhabited by the organism, etc.

A “transgenic animal” refers to any animal, a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of the gene. However, transgenic animals in which the recombinant gene is silent are also contemplated. Moreover, “transgenic animal” also includes those recombinant animals in which gene disruption of one or more genes is caused by human intervention, including both recombination and antisense techniques.

The term “heterologous,” when used with reference to a nucleic acid or polypeptide, indicates that a sequence that comprises two or more sequences which are not found in the same relationship to each other as normally found in nature, or is recombinantly engineered so that its level of expression, or physical relationship to other nucleic acids or other molecules in a cell, or structure, is not normally found in nature. For instance, a heterologous nucleic acid is typically recombinantly produced, having two or more sequences from unrelated genes arranged in a manner not found in nature; e.g., a nucleic acid open reading frame (ORF) of the invention operatively linked to a promoter sequence inserted into an expression cassette, e.g., a vector, of the invention. As another example, a polypeptide of the invention is linked to a tag, e.g., a detection- and purification-facilitating domain, as a fusion protein.

A “patient” is any mammalian individual, host, or subject, e.g., a human or a mouse.

A “modulator” is any substance, whether synthetic, semi-synthetic, or natural; organic or inorganic; small molecule or macromolecular; pharmaceutical, nucleic acid, or polypeptide, with the capability of altering a biological activity. The biological activity can be measured using any assay known in the art. An agent which modulates a biological activity of a subject polynucleotide or polypeptide increases or decreases the activity at least about 10%, at least about 15%, at least about 20%, at least about 25%, at least about 50%, at least about 100%, or at least about 2-fold, at least about 5-fold, or at least about 10-fold or more when compared to a suitable control. The term “modulate” encompasses an increase or a decrease, a stimulation, inhibition, or blockage in the measured activity when compared to a suitable control. “Modulation” of expression levels includes increasing the level and decreasing the level of an mRNA or polypeptide encoded by a polynucleotide of the invention when compared to a control lacking the agent being tested.

A “therapeutic” composition is one that is palliative, curative, or otherwise useful in treating or ameliorating a disease, disorder, syndrome or condition, or the recurrence of a disease, disorder, syndrome or condition.

A “pharmaceutically acceptable carrier” refers to a non-toxic solid, semisolid, or liquid filler, diluent, encapsulating material, or formulation auxiliary of any conventional type. A pharmaceutically acceptable carrier is non-toxic to recipients at the dosages and concentrations employed and is compatible with other ingredients of the formulation.

Structure of the Human HLJ1 Gene

Cloning and Sequence Analysis of the 5′ Flanking Region

A PCR-based strategy was used to isolate the promoter region of the human HLJ1 gene, as described in Example 1. A 5′ flanking region of 2,338 base pairs (bp) was obtained and subcloned into a promoterless pGL3-basic vector, producing the pGL3-HLJP construction. The identification of transcription factor binding sites in this region and the decrease in promoter activity upon deletion of this region in whole or in part (shown below) define this upstream portion of the HLJ1 gene as a promoter region. The nucleotide sequence of the HLJ1 promoter was submitted to the GenBank™ database with accession number AY669319 on Jun. 14, 2005 (68).

Bioinformatic Sequence Analysis of the Human HLJ1 Gene

Transcriptional elements of the HLJ1 gene promoter and its enhancer were identified and characterized by computer analysis, as described in Example 1. Sequence analysis using the ProScan program (http://bimas.dcrt.nih.gov/molbio/proscan) was used to predict CpG islands; it revealed no canonical TATA box (TATAAA) within the 1.0 kb region upstream of the transcription start site. ProScan detected an inverted CCAAT box (ATTGG) at position −247, although the latter is more frequently found at approximately position −70 in promoters lacking TATA boxes (21). GC-rich sequences are often reported in TATA-less promoters (22); this analysis of the HLJ1 promoter region revealed that it includes a GC box, at position −761.

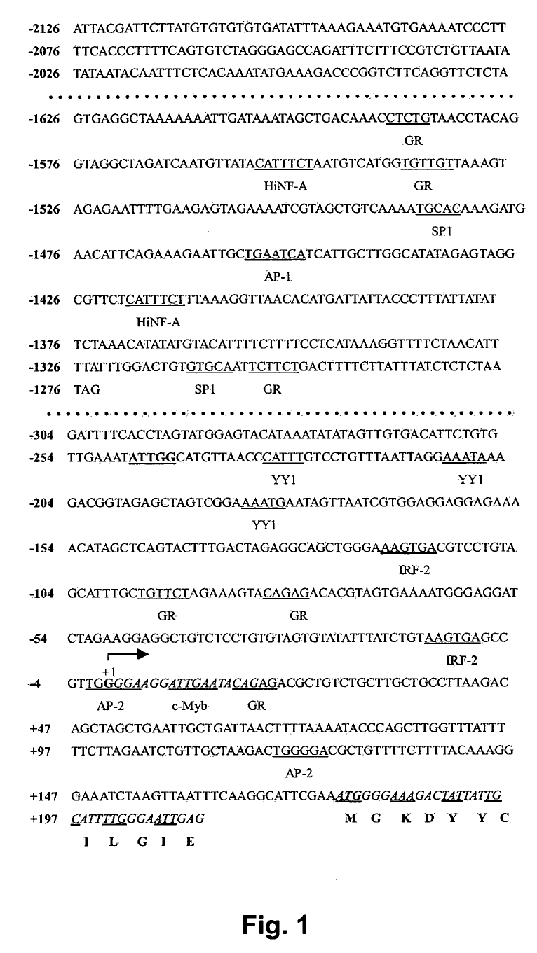

A computer search for potential regulatory elements in the 5′ flanking region using Matinspector V2.2 at the settings of core similarity 0.8 and matrix similarity 0.9 with the TRANSFAC database (16) revealed consensus sequences for transcription factor binding sites, including Yin Yang 1 (YY1), a common transcription factor often used in the absence of a TATA box; activated protein-1 (AP-1), which is comprised of the gene products of c-jun and c-fos proto-oncogenes, and has been reported to mediate the transcriptional down regulation of the matrix metalloprotease MMP-9 promoter by a conditionally activated c-fos fusion protein (32); stimulating protein-1 (SP1), which can bind to a GC box, and can initiate transcription of a variety of cellular and viral genes, including HIV; glucocorticoid receptor (GR); histone nuclear factor A (HiNF-A); activated protein-2 (AP-2); and interferon regulatory factor 2 (IRF-2) (FIG. 1).

Transcriptional Start Site of the Human HLJ1 Gene

To determine the transcriptional start site(s) of the human HLJ1 gene, 5′-RACE was performed as described in Example 2, using RNA isolated from the human adenocarcinoma cell line CL1-0. Following a secondary nested PCR reaction, the amplified fragment was isolated, subcloned, and sequenced. The resulting fragment demonstrated that the transcriptional start site was located 176 bp upstream of the translational initiation site (ATG) (FIG. 1). The cap switch method (17), as described in Example 2, also demonstrated that translation begins at nucleotide +176. Human HLJ1 Gene Transcriptional Activity

To examine human HLJ1 gene promoter activity in human adenocarcinoma cells, a series of promoter fragments with 5′-end deletions were generated by PCR and ligated to the pGL3-basic vector, as shown in FIG. 2 and further described in Example 3. The plasmids, comprising full-length, partially deleted, or entirely deleted HLJ1 upstream DNA and a luciferase reporter were transiently cotransfected with a β-galactosidase expression plasmid (pSV-β-Gal) into CL1-0 cells. Transfections were carried out in duplicate and individual experiments were repeated three times. Luciferase activity was measured in crude cell lysates and normalized to α-galactosidase activity and expressed relative to the pGL3-promoter vector. Promoter activity was expressed relative to a promoterless construct and normalized to β-galactosidase activity.

A plasmid containing the entire 1214 bp upstream of the initiation codon (pGL3-F2RER′) had higher luciferase activity in CL1-0 cells, resulting in an approximate 27-fold increase in luciferase activity as compared to the pGL3-basic vector (i.e., the negative control). Sequential deletion of 661 bp from the 5′-end of the HLJ1 promoter region (pGL3-F3RER′) resulted in almost the same promoter activity compared to the construct containing the entire 1214 bp region (−1038/+176). However, deleting nucleotides from −232 to −122 (pGL3-F6RER′) eliminated luciferase activity, leaving only control level activity for the plasmid containing the 298 nucleotides of the cloned region (−122/+176). The invention provides that YY1 transactivates the HLJ1 promoter by directly binding to the basal promoter and that YY1 requires the presence of its DNA-binding domain to transactivate the HLJ1 promoter, as shown by transfection experiments with reporter constructs having different truncations in the DNA-binding domain (68).

Enhancer Region, Core Promoter Region, and Silencing Element

HLJ1 enhancer (FIG. 2A) and promoter (FIG. 2B) constructs were prepared with the luciferase reporter gene as described in Example 3. The promoter activities of these constructs were compared following their transfection into CL1-0 cells. Transfections were performed as described in Example 4. The results shown represent the mean and standard deviation (SD) from at least three separate experiments. The error bars indicate the standard error of the mean (SEM). In FIG. 2B, the constructs on the left correspond with the bars quantifying luciferase activity levels on the right. Luciferase activity was measured in crude cell lysates, and reflects promoter activity. Promoter activity was expressed relative to a promoterless construct and normalized to the β-galactosidase activity of a co-transfected control, pSV-β-Gal.

The results shown in FIG. 2 demonstrate the presence of an enhancer region located from nucleotides −2,126 to −1,039 (FIG. 2A) and the presence of basal promoter activity between nucleotides −232 and −122 (FIG. 2B). As shown in FIG. 2A, the plasmid pGL3-FRER′, which contains 2,302 bp upstream of the initiation codon, beginning at nucleotide −2126 and including the 176 bp of transcribed, but untranslated, DNA, displayed a high level of promoter activity in CL1-0 cells. Its activity was approximately 500-fold greater than the negative control pGL3-basic vector, which is about the same level of activity of the strong SV40 viral promoter in CL1-0 cells.

Deleting 601 bp from the 5′ end of the HLJ1 promoter region resulted in a 50% decline in promoter activity compared to pGL3-FRER′. Further deletion of 5′ end sequences spanning nucleotides −1,039 to −232 resulted in decreased promoter activity, as demonstrated by the promoter activities of pGL3-F2RER′, pGL3-F3RER′, pGL3-F4RER′, and pGL3-F5RER′ (FIG. 2B). Deleting nucleotides −232 to −122 (pGL3-F6RER′) decreased promoter activity to control levels (FIG. 2B).

As shown in FIG. 2B, deletions in the regions spanning nucleotides −1,039 to −232 produced a dramatic decrease in promoter activity. The deletion constructs pGL3-F2RER′, pGL3-F3RER′, pGL3-F4RER′, pGL3-F5RER′, pGL3-F6RER′, and pGL3-F2REF/R′ showed about a 15 to about a 38-fold increase in promoter activity as compared to the pGL3-basic vector. Deleting nucleotides −232 to −122 (pGL3-F6RER′) abolished promoter activity. The pGL3-F6RER′ construct, which comprises 298 nucleotides of the cloned promoter region, displayed a level of activity corresponding to the negative control pGL3-basic vector. The core promoter activity of this region is similar in magnitude to the promoter activity obtained with the strong SV40 viral promoter in CL1-0 cells.

Another set of deletion mutants also demonstrated the presence of an enhancer region, and identified the minimal nucleotide sequence possessing enhancer function. As shown in FIG. 3, various lengths of the region between nucleotides −2,126 and −1,039 were subcloned into the enhancer-less pGL3-SV40-promoter vector to generate pGL3-p-EFR, pGL3-p-EF1 R, pGL3-p-EF2R, and pGL3-p-EF3R, as described in Example 3. CL1-0 cells were transiently transfected with each of these constructs and transcriptional activation was determined by measuring luciferase activity (FIG. 3). Nucleotides −2,126 and −1,039 efficiently increased transcription about 12-fold (pGL3-p-EFR) compared to the empty pGL3-promoter vector. Further stepwise removal of nucleotides between −1,843 and −1,255 from the 5′ end of the construct resulted in an appreciable drop in luciferase expression (pGL3-p-EF1 R, pGL3-p-EF2R, pGL3-p-EF3R). The* denotes a=0.05, P<0.05, as compared with the pGL3-p-EFR. The # denotes α=0.05, P<0.005, as compared with the pGL3-promoter vector control.

A bidirectional deletion of the putative enhancer region of vector pGL3-p-EF2R1 demonstrated that a 369 nucleotide fragment was able to support transcriptional activity at a level 2-fold greater than the entire 1,087 bp of the enhancer region (pGL3-p-EFR) (a=0.05, P=0.032), and 19-fold greater than the empty pGL3-promoter vector (a=0.05, P=0.003) (FIG. 3). This 369-nucleotide region, embodied in pGL3-p-EF2R1, is, therefore, the minimal enhancer domain of the HLJ1 gene enhancer region. It includes several potential binding sites for the transcription factors Window_{—}4, AP-1, SP1, NF-E2, and GR.

Stepwise removal of nucleotides between −1,843 and −1,255 from the 3′ end of the enhancer region produced an appreciable drop in HLJ1 gene transcription (FIG. 3). The construct pGL3-p-EFR1, which has a 3′-end deletion of the enhancer region, demonstrated the highest enhancer activity. Further 3′-end deletion constructs (pGL3-p-EF2R and pGL3-p-EFR3) demonstrated markedly lower enhancer activity (FIG. 3). This demonstrates the presence of a silencing element in the region of nucleotides −1,843 to −1,255.

A positional effect between the enhancer and basal promoter regions is demonstrated in FIG. 4. Luciferase activity was measured in CL1-0 cells transiently transfected with the constructs shown in FIG. 4. Transfections were carried out in duplicate and individual experiments were repeated three times. Variation within an individual experiment was controlled by co-transfecting with the pSV-β-Gal vector. A recombinant enhancer-promoter construct with nucleotides −1,039 to −232 subcloned into the pGL3-basic vector, pGL3-EFR-F5RER′, efficiently drove luciferase expression at a level about 18-fold greater than the HLJ1 basal promoter construct pGL3-F5RER′. Recombinant pGL3-EFR—F5RER′ possessed 36% of the promoter activity of the 2,302 bp full-length HLJ1 promoter (pGL3-FRER′). Recombinant pGL3-EFR1—F5RER′, with the enhancer region nucleotides −2,126 to −1,255, possessed about the same amount of promoter activity as pGL3-EFR—F5RER′, demonstrating that the silencing element is regulated by a positional effect with respect to its relationship to the basal promoter region.

FIG. 4 also demonstrates a silencing element located in the 216 nucleotides at the 3′-end (positions −1,255 to −1,039) of pGL3-p-EFR. Deleting this region increased enhancer activity about 3-fold compared to pGL3-p-EFR activity. Deleting additional nucleotides from the 3′ end resulted in a dramatic fall in enhancer activity, as demonstrated by pGL3-EFR—F5RER′, pGL3-EFR1—F5RER′ and pGL3-EF2R1-F5RER′. The recombinant construct pGL3-EF2R1-F5RER′ demonstrated 45% of the promoter activity of pGL3-EFR—F5RER′, and 16% of the promoter activity of the full-length HLJ1 promoter (pGL3-FRER′).

Also as demonstrated in FIG. 4, nucleotides −2126 to −1039 of the human HLJ1 gene have a positive effect on promoter activity, and therefore constitute an enhancer region. Deleting the region between −2,126 and −1526 resulted in a 50% decrease in promoter activity compared to the activity of the pGL3-FRER′ construct. Deletion of the region between −2,126 and −1,039 resulted in a nearly complete loss of promoter activity, as compared to pGL3-FRER′ (α=0.05, P=0.004). Promoter activity analysis also demonstrates a core promoter region of the HLJ1 gene between nucleotides −232 and +176 (pGL3-F5RER′ construct, FIG. 4).

Human HLJ1 Gene Promoter Mediates Cell Specific Expression

The human lung adenocarcinoma cell lines CL1-0 and CL1-5 differ both in their level of endogenous HLJ1 expression and their metastatic ability. CL1-0 cells express high levels of HLJ1 and have low metastatic activity, whereas CL1-5 cells express low levels of HLJ1 and have high metastatic activity (13). As shown in FIG. 5, transiently transfected CL1-0 and CL1-5 cells displayed different levels of HLJ1 when transfected with the same construct.

As shown in FIG. 5A, CL1-0 cells transfected with deletion constructs having functional promoter regions displayed stronger promoter activity than CL1-5 cells transfected with the same constructs. HJL1 gene expression in CL1-0 and CL1-5 was compared by Northern Blot analysis as described in Example 5. As shown in FIG. 5B, two major HLJ1 transcripts of approximately 2.5 and 3.6 kb were observed. HLJ1 gene expression was observed to be higher in the less metastatic CL1-0 cells than the more metastatic CL1-5 cells. The strength of the promoter activity in the less metastatic CL1-0 cells was about 10-fold higher than in the highly metastatic CL1-5 cells.

Transcriptional Activity of HLJ1 in Lung Cancer

YY1 Expression Increases HLJ1 Expression and Reduces CL1-0 Motility

YY1 is a 65-kDa multifunctional zinc finger transcription factor that belongs to the human GLI-Kruppel family of nuclear proteins (23-28). It has been shown to bind to the specific DNA consensus sequence 5′-CGCCATNTT-3′, which is present in many promoters, and can regulate transcriptional activity by either activation or repression (23, 29, 30). Both the promoter context and the cellular environment influence YY1 activation (24, 26, 31).

As shown in FIG. 6A, expressing the pcDNA3-YY1 construct in the presence of pGL3-F5RER′ increased promoter activity in a dose-dependent manner. HLJ1 promoter constructs and pcDNA3-YY1 constructs were co-transfected into CL1-0 cells and promoter activity was measured and expressed as relative luciferase activity.

As shown in FIG. 6B, this result was not observed when pcDNA-YY1 was expressed, not in the presence of pGL3-F5RER′, but rather, in the presence of the control vectors pGL3-basic or pGL3-F6RER′, which lack both YY1 binding sites and promoter activity. Thus, HLJ1 basal promoter activity is positively correlated with levels of co-transfected pcDNA3-YY1 construct. The transcription factor YY1 is shown herein to regulate HLJ1 gene promoter activity in a dose-dependent manner. Endogenous YY1 expression was observed to be similar in CL1-0 and CL1-5 cells.

The role of YY1 in regulating HLJ1 gene expression is further demonstrated by the Western Blot shown in FIG. 7 and further described in Examples 7 and 8. CL1-0 cells were transfected with the pcDNA3-YY1 construct, PCY3-1, PCY4-2, and PCY4-5 were tested for protein expression. All three clones expressed higher levels of both YY1 protein and HLJ1 protein than control cells transfected with empty pcDNA3 vector (PCC1), or untransfected control CL1-0 cells, demonstrating that YY1 expression correlates with translation of the HLJ1 gene into protein.

HLJ1 Suppresses Cancer Cell Motility

The directional migration of CL1 cells transfected with the pcDNA3-YY1 construct was blunted compared to their untransfected counterparts. The three stably transfected CL1 clones, PCY3-1, PCY4-2, and PCY4-5, were examined using a scratch wound assay. The cells were seeded into culture plates at a concentration of 2.5×10⁵ cells/well and wounded as described in Example 9. After wounding, the cultures were incubated at 37° C. and photographed immediately (0 h), 24 h, and 48 h later. A duplicate culture was stained with crystal violet and photographed at 72 h. Migration was evaluated by the number of cells migrating into the cell-free zone. The experiments were repeated in quadruplicate wells at least three times.

As shown in FIG. 8, YY1 expression decreased cell motility. The directional migration of cells in the untransfected CL1-0 cell monolayer into the wound was compared to stably transfected and mock transfected cells by counting the number of cells that migrated into the wounded area, i.e., the area between the pairs of dotted lines shown in FIG. 8. The YY1-transfected CL1-0 cell clones (PCY3-1, PCY4-2, and PCY4-5) migrated into the wound at a slower rate than untransfected CL1-0 cells and mock-transfected cells (PCC2). YY1-transfection reduced cell migration activity to about 50% (PCY3-1), 43% (PCY4-2), and 38% (PCY4-5) of the levels of untransfected CL1-0 cells. FIG. 8 shows a representative example of three similar experiments performed on independently transfected groups of cells.

Differential Expression of HLJ1 in CL1 Cells

HLJ1 expression was examined in each of a panel of lung cancer cell lines including CL1-0, CL1-1, CL1-5, and CL1-5-F4, listed in order of increasing metastatic ability (FIG. 9A). HLJ1 expression was negatively correlated with the metastatic ability of these lung cancer cell lines. As shown in FIG. 9A, HLJ1 gene expression was highest in CL1-0 cells, diminished in CL1-1 and CL1-5 cells, and the lowest level of HLJ1 expression was observed in the most highly metastatic CL1-5-F4 cells.

Differential expression of the HLJ1 gene in these model cell lines was confirmed by determination of the steady-state HLJ1 mRNA level by Northern blot analysis as described in Example 7. As shown in FIG. 9B, Northern blot analysis revealed the two major HLJ1 transcripts of approximately 2.5 and 3.6 kb. The level of HLJ1 RNA expression was consistent with the results of the microarray, as described in Example 10. CL1-0 cells expressed a high level of HLJ1 mRNA, and progressively less mRNA was expressed by CL1-1, CL1-5, and CL1-5F4 cells.

As shown in FIG. 9C, RTQ RT-PCR analysis of HLJ1 gene expression performed as described in Example 11 further demonstrated that HLJ1 gene expression correlated with metastatic ability. All cell lines examined expressed HLJ1 mRNA; the expression level was estimated to be approximately 8-fold higher in the less invasive CL1-0 and CL1-1 cells than in the highly invasive CL1-5 and CL1-5-F4 cells.

As shown in FIG. 9D, the correlation of HLJ1 gene expression and metastatic ability extends to the expression of HLJ1 protein. Analysis of HLJ1 protein levels by Western blotting are consistent with the expression pattern of HLJ1 mRNA shown in FIGS. 9A, 9B, and 9C. HLJ1 protein is more highly expressed in C1-0 and CL1-1 cells than in the more highly metastatic CL1-5 and CL1-5F4 cells. Taken together, FIGS. 9A-D demonstrate that the expression of the HLJ1 gene is negatively correlated with the cell's metastatic ability.

Effect of Heat Shock on HLJ1 Expression in CL1-0 and CL1-5 Cells

CL1-0 and CL1-5 cells were subjected to a heat shock as described in Example 6. The mRNA was then extracted, and the expression level of HLJ1 was estimated by Northern blot analysis as described in Example 5 and RTQ RT-PCR as described in Example 11. As shown in FIG. 10, HLJ1 mRNA expression was increased approximately 2-fold in CL1-0 cells by heat shock treatment, but the heat shock response was lost or delayed in CL1-5 cells (FIG. 10A). In a similar manner, HLJ1 mRNA expression was increased approximately 2-fold in CL1-0 cells by heat shock treatment, but the heat shock response was lost or delayed in CL1-5 cells (FIG. 10B).

Subcellular Localization of HLJ1 Protein

The intracellular localization of HLJ1 protein was examined in CL1-5 cells transfected with the mammalian transfection vector pEGFP-C3, as described in Example 12. Fluorescent images, obtained by laser scanning confocal microscopy, showed that EGFP-tagged HLJ1 protein was distributed in the nucleus, and especially enriched in the nucleoli (FIG. 11).

HLJ1 Reduces Cell Proliferation and Anchorage-independent Growth

The highly invasive lung adenocarcinoma cells CL1-5, which have low levels of endogenous HLJ1 expression, were stably transfected with pCDNA3-HLJ1 plasmid containing full-length HLJ1 cDNA (FIG. 12). The HLJ1 expression level in CL1-5 cells, PCC10 cells, which are the transfection controls that are transfected with empty vector, and PCH9 and PCH12, which are the stable HLJ1 transfectants, were evaluated using RTQ RT-PCR as described in Example 11. As shown in FIG. 12A, HLJ1 mRNA expression was increased in cells transfected with HLJ1 as compared to mock-transfected or untransfected cells. As shown in FIG. 12B, HLJ1 protein expression was increased in cells transfected with HLJ1 as compared to mock-transfected or untransfected cells. Statistically significant differences were observed by both RTQ RT-PCR and Western blot when each transfected clone was compared to the CL1-5 cells (p<0.05 by student's t-test).

The growth rates of these stable transfectants were measured by the MTT cell proliferation assay, as described in Example 13. Cells were grown in log phase for 1 to 4 days and reached confluence by days 5 or 6. FIG. 12C shows that the proliferation rate of stable transfectants PCH9 and PCH12 was decreased significantly as compared to the proliferation rate of the control transfectants PCC10 and the parental cell line CL1-5. Each experiment was performed at least three times and similar results obtained each time.

The anti-metastatic potential of these stable transfectants was measured in vitro by a soft agar assay for anchorage-independent growth, as described in Example 14. After plating 6×10³ cells in triplicate in soft agar, the number of colonies formed and the colony size were analyzed after 3 weeks. Colonies greater than 1 mm were scored as positive. FIG. 12D demonstrates that CL1-5 cells and PCC10 cells possess a greater potential for anchorage independent growth, as reflected by their ability to form colonies in soft agar, compared to the PCH9 HLJ1 stable transfectants, which were less capable of colony formation (p<0.01). The top row of FIG. 12D shows cells growing in soft agar on tissue culture plates following crystal violet staining. The bottom row shows the number of colonies on each plate. Plating more cells or lengthening the incubation period did not increase PCH9 colony formation. Taken together, these data from FIG. 12 demonstrate that HLJ1 expression decreases cell proliferation.

Expression of HLJ1 Suppresses In Vitro Invasion and Migration

The metastatic potential of tumors depends, in part, on the ability of the tumor cells to invade through a basement membrane and migrate to distant sites. The ability of cells transfected with HLJ1 to penetrate a Matrigel™ membrane using an in vitro assay, which is a modified Boyden chamber assay, as described in Example 15, was determined as an index of the metastatic potential of these cells. The YY1 transfectant PCY3, which expressed a high level of HLJ1, reduced cell invasion capability to about 56% and about 61% of the levels of CL1-0 and mock transfectants, respectively (68).

As shown in FIG. 13, the invasive potential of CL1-5/HLJ1 transfected cells was lower than their untransfected counterparts. Inhibiting the ability of transfected cells to express HLJ1 can restore their invasive capacity. Small inhibiting RNA (siRNA) specific for HLJ1 decreased the endogenous HLJ1 RNA level in the YY1 transfected cell line, PCY3. These siRNAs also increased the ability of PCY3 cells to invade through Matrigel™.

Two siRNA sequences were synthesized according to standard protocols (68). The sequence of HLJ1-A was AACCCGGMTGAGGAGAAGAA. The sequence of HLJ1-D was AAACGCTGATGGAAGGAGTTA. HLJ1-A and HLJ1-D reduced its expression by 64%. HLJ1-A and JLJ1-D restored the ability of PCY3 cells to invade through Matrigel™ to 75% (p=0.02) and 87% (p<0.005), compared to PCC2 cells, which do not express HLJ1. A negative control, scrambled siRNA, had no significant effect on HLJ1 expression or invasive capacity.

After 16 h in the Matrigel™ chamber, significantly fewer (40%-60%) HLJ1 transfectants than control cells had invaded the membrane. As shown in FIG. 13A, the incidence of invasion was higher after 24 hr and 48 hr in the Matrigel™ chamber in untransfected CL1-5 and mock-transfected PCC10 control cells than in the stable transfectants PCH9 and PCH12, which overexpress HLJ1, demonstrating that HLJ1 reduces the invasiveness of metastatic cells.

The metastatic potential of tumors depends, in part, also, on the ability of the tumor cells to undergo directional migration, such as into a wound. The ability of HLJ1 transfectants to migrate across a scratch wound was assayed to determine their migration ability. Confluent monolayers of CL1-5, PCC10, and PCH9 cells were scratch-wounded as described above, and their migratory capability assayed as described in Example 9. The migration of PCH9 cells was markedly suppressed (up to 60%) compared with CL1-5, PCC10 cells, as shown by the photographs of cells in the upper panel of FIG. 13B, and the bar graph in the lower panel of FIG. 13B showing the reduction in the number of PCH9 cells that migrated into the wound, expressed as a percentage of the control CL1-5 cells. Taken together, FIGS. 13A and B demonstrate that HLJ1 inhibited the migration of metastatic cells.

HLJ1 Inhibits In Vivo Tumor Growth

The effect of HLJ1 expression on the tumorigenicity of CL1-5 cells in vivo was determined in severe combined immunodeficiency syndrome (SCID) mice, as described in Example 16. SCID mice have defects in the development of their immune systems, and as a result, allow disseminated growth of human tumors. Exponentially growing lung adenocarcinoma cells were injected into 6 week-old SCID mice; PCH9 cells were injected into the right side and PCC10 cells were injected into the left side of each of six mice. HLJ1 expression resulted in marked inhibition of tumor growth in SCID mice (FIG. 14A). The PCH9 cells failed to develop tumors (six out of six) three weeks after inoculation, while the PCC10 controls developed tumors (six out of six).

After three weeks, the PCH9 adenocarcinoma cells began to form tumors, as shown in FIG. 14(B). A comparison of the size of the tumors formed by PCH9 and PCC10 cells is shown in FIG. 14B. At three weeks, all six of the PCC10 tumors were larger than 1000 mm³. At five weeks, the tumors resulting from the PCH9 cells reached only approximately 100 mm³ in size, whereas the tumors from the PCC10 cells were approximately 5000 mm³. Therefore, HLJ1 expression reduced both tumor incidence and tumor growth rate in an in vivo model. HLJ1 mRNA Expression in Lung Adenocarcinoma Patients

Semiquantitative RT-PCR was used to determine HLJ1 expression levels in human lung cancer tissue and adjacent normal lung tissue from 43 patients with lung adenocarcinoma, as described in Example 17. Resolution of the PCR products on a 1% agarose gel demonstrated that expression of HLJ1 mRNA in all ten tumor specimens examined was significantly lower than in the adjacent normal tissue (FIG. 15). Therefore, HLJ1 gene expression is predictive of and diagnostic for cancer.

To quantify HLJ1 transcript levels, RTQ RT-PCR was used to determine numbers of HLJ1 transcripts in lung cancer tissue and adjacent normal lung tissue in the same patients, who were classified into high-expression or low-expression groups. The mean value of HLJ1 expression levels was used to delineate these groups. Survival curves (FIG. 16A) and relapse curves (FIG. 16B) were obtained by the Kaplan-Meier method as described in Example 19, and the difference in survival and relapse time between groups with low and high expression of HLJ1 was analyzed with the log-rank test. The Kaplan-Meier method The results showed that reduced expression of HLJ1 was statistically significantly associated with early postoperative relapse (p=0.0202) and shorter survival (p=0.0059) in lung adenocarcinoma patients (FIG. 16).

It must be noted that, as used herein, the singular forms “a,” “or,” and “the” include plural referents unless the context clearly dictates otherwise. Thus, for example, reference to “an isolated nucleic acid molecule” includes a plurality of such molecules and reference to “a modulator” includes reference to one or more modulators and equivalents thereof known to those skilled in the art.

Further, all numbers expressing quantities of ingredients, reaction conditions, % purity, polypeptide and polynucleotide lengths, and so forth, used herein, are modified by the term “about,” unless otherwise indicated. Accordingly, the numerical parameters set forth herein are approximations that may vary depending upon the desired properties of the present invention. At the very least, and not as an attempt to limit the application of the doctrine of equivalents to the scope of the claims, each numerical parameter should at least be construed in light of the number of reported significant digits, applying ordinary rounding techniques. Nonetheless, the numerical values set forth in the specific examples are reported as precisely as possible. Any numerical value, however, inherently contains certain errors from the standard deviation of its experimental measurement.

With respect to ranges of values, the invention encompasses each intervening value between the upper and lower limits of the range to at least a tenth of the lower limit's unit, unless the context clearly indicates otherwise. Further, the invention encompasses any other stated intervening values. Moreover, the invention also encompasses ranges excluding either or both of the upper and lower limits of the range, unless specifically excluded from the stated range.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Cloning and Analysis of the Human HLJ1 5′ Flanking Region

A PCR based method was used to clone the 5′ flanking region of the human HLJ1 gene. Specific primers were designed from the 5′-end of the known HLJ1 cDNA sequence (7) and from GenBank. CL1-0 cell genomic DNA was isolated by QIAamp DNA blood mini kit (Qiagen) and served as the PCR template. The sequences of the primers in the primer set used in the PCR amplification are: HLJP-F primer, 5′-CCGCTCGAGATTACGATTCTTATGTGTG TG-3′ (SEQ. ID. NO.:37), which introduced an XhoI site (underlined); and HLJP-R1 primer, 5′-CCCAAGCTTCTCAATTCCCAAAAT GCAAT AATAG-3′ (SEQ. ID. NO.:38), which introduced a HindIII site (underlined). The PCR conditions were as follows: one cycle for 2 min 30 sec at 94° C., 1 min at 55° C., 3 min at 72° C.; followed by 34 cycles for 40 sec at 94° C., 1 min at 60° C., 3 min at 72° C.; and final extension at 72° C. for 10 min. The amplified DNA fragment consisted of 2,338 bp; it was digested with XhoI/HindIII and cloned into the promoterless pGL3-basic (Promega, Madison Wis.) vector to produce pGL3-HLJP. The fragment was sequenced and found to be contiguous with the HLJ1 cDNA.

Homology searches were performed on this 2,338 bp fragment, which is shown, in part, in FIG. 1, using Basic Local Alignment Search Tool (BLAST) from the National Center for Biotechnology Information (NCBI) (http://www.ncbi.nim.nih.gov)

(69). Putative transcription factor binding elements in the HLJ1 promoter were detected and analyzed using the programs Matinspector 2.2 (14) and SignalScan (15), which are available through the Bioinformatics and Molecular Analysis Section of the United States Institutes of Health; http://thr.cit.nih.gov/molbio/signal/, Genomatix Software GmbH; http://www.genomatix.de, and the TRANSFAC database (16). The programs Grail 1.3 (http://compbio.oml.gov/grail-1.3) (70) and ProScan (http://bimas.dcrt.nih.gov/mol bio/proscan) (71) were used to predict CpG islands and promoter regions.

### Example 2

### 5′-Rapid Amplification of cDNA Ends (RACE)

Transcription start sites were identified by 5′-rapid amplification of cDNA ends using the 5′-RACE method as previously described (17). Briefly, 10 μg total RNA isolated from CL1-0 cells was reverse transcribed by Superscript RT II (Gibco Life Technologies (Invitrogen Life Technologies), Carlsbad, Calif.) using the T20 primer (5′-TTTTTTTTTTTTTTTTTTTT-3′) and the CapSwitch primer (5′-AAGCAGTGGTATCAA CGCAGAGTACGCrGrGrG-3′). The reverse transcription PCR was first performed on a DNA cycler at 42° C. for 1 hour and 94° C. for 5 minutes. One μl of the first-stranded cDNA was added to 50 μl PCR mixture with TSP primer (5′-GCAGTGGTATC AACGCAGAG-3′) and QHLJ1-R primer (5′-CCATCCAGTGTTGGTACATTAATT-3′) (SEQ. ID. NO.:39). The PCR conditions were one cycle for 2 min 30 sec at 94° C., 1 min at 55° C. and 3 min at 72° C.; followed by 34 cycles for 40 sec at 94° C., 1 min at 60° C., and 3 min at 72° C.; then a final extension step at 72° C. for 10 min. The reverse transcription PCR products were separated on a 1% agarose gel and the 5′-RACE products were purified and subcloned in to PCRII TOPO vector (Invitrogen Corp., Carlsbad, Calif.) according to the manufacturer's instructions, then sequenced.

### Example 3

### Construction of Luciferase Reporter Gene Constructs

Promoter constructs corresponding to varying lengths of the 5′ flanking region of the HLJ1 gene were generated by PCR using the pGL3-HLJP construct as the template. A common reverse primer (HLJP-RER) (SEQ. ID. NO.:22) and different forward primers (HLJP-F1, HLJP-F2, HLJP-F3, HLJP-F4, HLJP-F5, HLJP-F6, and HLJP-REF) (SEQ. ID. NO.:15-21), shown in Table 1, were used to amplify various deletion fragments, producing the promoter constructs. XhoI and HincII sites were introduced into the forward and reverse primers, respectively, and used to clone these fragments upstream of a luciferase reporter gene in the promoterless vector pGL3-basic (Promega, Madison Wis.). The pGL3-Control plasmid was used as a positive control, and was also obtained from Promega (Madison Wis.).

A similar approach was used to make the HLJ1 enhancer constructs, which were also generated by PCR and ligated into the MluI/XhoI sites of the enhancerless pGL3-Promoter (Promega, Madison Wis.) vector. The forward and reverse PCR primers (SEQ. ID. NO.:23-SEQ. ID. NO.:36) used to amplify the HLJ1 enhancer clones are also listed in Table 1. The composition of all of the constructs was confirmed by restriction endonuclease digestion and DNA sequencing.

TABLE 1

Primer sequences used to amplify HLJ1
promoter and enhancer constructs

| | Amplification | |
|---|---|---|
| SEQ. ID. NO. | primer | Primer Sequence (5′ to 3 ) |
| | Promoter forward | |
| | primers | |
| SEQ. ID. NO.:15 | HLJP-F1 | CCGCTCGAGAATTTTGAAGAGTAGAAAATCGTA |
| SEQ. ID. NO.:16 | HLJP-F2 | CCGCTCGAGGGATTACCTAAAATGATATTATAGG |
| SEQ. ID. NO.:17 | HLJP-F3 | CCGCTCGAGTAGAATTGTCGTTCCTTTTATCTGT |
| SEQ. ID. NO.:18 | HLJP-F4 | CCGCTCGAGATTTTCTCCTAGTATGGAGTACATA |
| SEQ. ID. NO.:19 | HLJP-F5 | CCGCTCGAGCATTTGTCCTGTTTAATTAGGAAA |
| SEQ. ID. NO.:20 | HLJP-F6 | CCGCTCGAGGGAAAGTGACGTCCTGTA |
| SEQ. ID. NO.:21 | HLJP-REF | CCGCTCGAGGGGAAGGATTGAATACAGA |
| | Promoter reverse | |
| | primer | |
| SEQ. ID. NO.:22 | HLJP-RER | CCCAAGCTTTTCGAATGCCTTGAAATTAAC |
| | Enhancer forward | |
| | primers | |
| SEQ. ID. NO.:23 | HLJP-EF | CGACGCGTATTACGATTCTTATGTGTGTG |
| SEQ. ID. NO.:24 | HLJP-EF1 | CGACGCGTAGAACAATTTCCGGTT |
| SEQ. ID. NO.:25 | HLJP-EF2 | CGACGCGTTTGATATTATTTCTTGGTGA |
| SEQ. ID. NO.:26 | HLJP-EF3 | CGACGCGTTTCTTATTTATCTCTCTAATAG |
| SEQ. ID. NO.:27 | HLJP-EF21 | CGACGCGTCCTCTGTAACCTACAGGTAG |
| SEQ. ID. NO.:28 | HLJP-EF22 | CGACGCGTATGGTGTTGTTAAAGTAGAGA |
| SEQ. ID. NO.:29 | HLJP-EF23 | CGACGCGTAAAATGCACAAAGATGAACAT |
| SEQ. ID. NO.:30 | HLJP-EF24 | CGACGCGTTGGCATATAGAGTAGGCGTT |
| SEQ. ID. NO.:31 | HLJP-EF25 | CGACGCGTTTACCCTTTATTATATTCTAAACA |
| SEQ. ID. NO.:32 | HLJP-EF26 | CGACGCGTAAGGTTTTCTAACATTTTATTTG |
| | Enhancer reverse | |
| | primers | |
| SEQ. ID. NO.:33 | HLJP-ER | CCGCTCGAGCCTATAATATCATTTTAGGTA |
| SEQ. ID. NO.:34 | HLJP-ER1 | CCGCTCGAGCTATTAGAGAGATAAATAAGAAAAGTCA |
| SEQ. ID. NO.:35 | HLJP-ER2 | CCGCTCGAGTCACCAAGAAATAATATCAA |
| SEQ. ID. NO.:36 | HLJP-ER3 | CCGCTCGAGAACCGGAAATTGTTCT |

^{a} Restriction enzyme cutting sites used in PCR primers are underlined. XhoI site: CTCGAG; HindIlI site: AAGCTT; MluI site: ACGCGT.

The expression vectors were constructed from total RNA isolated from CL1-0 cells using Trizol reagent (Life Technologies, Inc., Gaithersburg, Md.). First-strand cDNA was reverse transcribed with SuperScript II reverse transcriptase (Life Technologies, Inc., Gaithersburg, Md.) and an oligo-dT primer. The HLJ1 coding region (GenBank accession number NM_{—}007034) was amplified by polymerase chain reaction (PCR) using the following forward and reverse primers: the forward primer 5′-CGCGGATCCATGGGGAAA GACTATTATTGC-3′ (SEQ. ID. NO.:40), which introduced an BamHI site (underlined), and the reverse primer 5′-GCTCTAGAATTCTATGAGG CAGGAAGATG-3′ (SEQ. ID. NO.:41), which introduced an XbaI site (underlined), under the following conditions: denaturing for 1 min at 94° C., annealing for 1 min at 55° C., and elongation for 2 min at 72° C. for 35 cycles. The amplified product was cloned into a pGEM-T Easy vector (Promega, Madison, Wis., USA). The coding region of HLJ1 cDNA was excised by BamHI/XbaI and subcloned into the BamHI/XbaI site of the constitutive mammalian expression vector pCDNA3, which contains the cytomegalovirus enhancer-promoter (Invitrogen Corp., Carlsbad, Calif.). The cDNA was then fully sequenced to ensure that no mutations were introduced during the PCR amplification. The resulting plasmid construct was named pCDNA3-HLJ1.

Subsequently, CL1-5 cells were seeded into 6-cm dishes at 5×10⁵ cells/dish and transfected with pCDNA3-HLJ1 and pCDNA3 (empty vector) using Lipofectamine transfection reagent (Invitrogen Corp., Carlsbad, Calif.) according to the manufacturer's protocol. After culturing in medium containing 400 μg/ml Geneticin (G418; Invitrogen Corp., Carlsbad, Calif.) for 2-3 weeks, individual clones were isolated using cloning cylinders. The cell clones that expressed the HLJ1 cDNA coding region were maintained in medium containing 400 μg/ml of Geneticin and used for further investigation.

### Example 4

### Transfection and Luciferase Assays

Transfections were performed in triplicate in 6-well plates. Approximately 2×10⁵ cells/well were seeded 24 hours prior to transfection. Plasmids were transfected into cells using Lipofectamine reagent according to the manufacturer's instructions (Invitrogen Corp., Carlsbad, Calif.). The luciferase reporter constructs described in Example 2, along with a control plasmid, were cotransfected with a β-galactosidase construct, pSV β-Gal (Promega, Madison Wis.). The ratio of the DNA in the luciferase reporter constructs to the β-galactosidase constructs was 3:1. The cells were incubated in the manufacturer's transfection mixture for 4 h, then harvested after 44 h in culture.

Stable transfection experiments were performed with the YY1 expression plasmid (68) transfected into CL1-0 cells using Lipofectamine reagent and selected for growth in G418 (400 μg/ml). Co-transfection experiments were performed with a total of 11 μg of DNA. The reaction mixtures contained 10 μg of HLJ1 promoter-reporter luciferase plasmid and pGL3-basic vector DNA or YY1 expression plasmid in various ratios, plus 1 μg of internal control pSV β-Gal plasmid. An aliquot of cell lysate (10-25 μl) was used to assay luciferase activity using a Luciferase assay kit (Tropix, Inc, Bedford, Mass.). Another aliquot of cell lysate (10-25 μl) was used to measure β-galactosidase activity using the Galacto-Light chemiluminescent assay kit (Tropix, Inc, Bedford, Mass.). Luminescence was measured with a Victor² 1420 Multilabel Counter (Wallac). The transfection efficiency was normalized with β-gal activity. Each experiment was performed at least three times.

### Example 5

### Northern Blot Analysis

Northern blot analysis was performed using previously described procedures (35). The RNA in each lane was measured by comparing its signal intensity with that of the GAPDH probe (68), which was used as an internal control for RNA quantity.

Briefly, 2 μg mRNA were size-separated on 1% agarose formaldehyde gels, transferred onto a positively charged Hybond-N⁺ nylon membrane (Amersham Life Sciences, Arlington Heights, Ill.), and fixed by cross-linking with ultraviolet light. HLJ1 expression was detected by a Dig-11-dUTP labeled HLJ1 cDNA probe (68). The hybridization and washing procedures were carried out using standard protocols. Equal loading was confirmed and transfer efficiency was assessed by hybridizing the blots with a Dig-11-dUTP labeled GAPDH cDNA probe.

### Example 6

### Cell Culture and Heat-Shock Treatment

The human lung adenocarcinoma cell lines CL1-0 and CL1-5 were maintained at 37° C. in a humidified atmosphere of 5% CO₂ (55). Cells were cultured in RPMI 1640 medium (Life Technologies, Inc., Gaithersburg, Md.) with 10% heat-inactivated fetal bovine serum (FBS) (Life Technologies, Inc., Gaithersburg, Md.) and 1% penicillin streptomycin (Life Technologies, Inc., Gaithersburg, Md.).

The human lung adenocarcinoma cell lines CL1-0, CL1-1, CL1-5, and CL1-5-F4, listed in ascending order of invasive competence, were established as previously described (13, 55). Cells were cultured in RPMI-1640 medium (Life Technologies, Inc. Gaithersburg, Md.) with 10% heat inactivated fetal bovine serum (Life Technologies, Gaithersburg, Md.) and penicillin/streptomycin (100 mg/ml each) at 37° C. in a humidified atmosphere of 5% CO₂. For heat-shock treatment, CL1-0 and CL1-5 cells were first grown at 37° C. and then were shifted to 45° C. and incubated for 30 min. After heat-shock treatment, these cells were harvested immediately for RNA extraction. Control cells were maintained at 37° C.

### Example 7

### Western Blot Analysis

The details of nuclear extract preparation and their analysis by Western blot have been described previously (18). Total cell lysates were isolated from cells as described previously (34). HLJ1 and YY1 were detected using a 1:1500 dilution of mouse polyclonal anti-HLJ1 and a 1:1000 dilution of mouse monoclonal anti-YY1 primary antibodies (Santa Cruz Biotechnology, Inc., Santa Cruz, Calif.). Alpha-tubulin and TATA box binding protein (TBP), used as the internal gel controls, were detected using commercially available mouse monoclonal anti-α-tubulin or anti-TBP primary antibodies, respectively (Santa Cruz Biotechnology, Inc., Santa Cruz, Calif.).

Cells were harvested for total cell lysates with RIPA buffer (1% Nonidet P-40, 0.5% sodium deoxycholate, 0.1% SDS, 50 mM Tris-HCl, pH 7.5) containing protease inhibitors. Cell lysates were centrifuged at 13,000 rpm for 10 min at 4° C. The supernatant was collected, and the protein concentration was measured. The same amount of protein was added to each lane, resolved on a 10% SDS-polyacrylamide gel by electrophoresis, and transferred onto nitrocellulose membranes (Hybond TM-C Super, Amersham, Buckinghamshire, UK). The membranes were blocked in TBST (0.2 M NaCl; 10 mM Tris, pH 7.4; 0.2% Tween-20) containing 5% skim milk and then incubated with HLJ1 primary antibody in TBST containing 5% skim milk. The membranes were then incubated with horseradish peroxidase-conjugated goat anti-mouse secondary antibody (Santa Cruz Biotechnology, Inc., Santa Cruz, Calif.) in TBST containing 2% skim milk. Bound antibody was detected with an enhanced chemiluminescence system (ECL, Amersham, Arlington Heights, Ill.) and autoradiographed with Kodak X-omat AR film.

### Example 8

### Antibody Production

A His-tagged HLJ1 fusion protein was expressed in bacteria using the QIAexpressionist system (Qiagen, Valencia, Calif.). A 1.2 kb fragment of HLJ1 cDNA was excised from pGEM-HLJ1 and subcloned into the pQE30 producing an inducible expression vector coding for a His-tagged HLJ1 protein. Subsequently, the recombinant plasmids were transformed into Escherichia coli JM109 cells to produce N-terminal His-tagged HLJ1. The fusion protein expression was induced with 0.2 mg/ml IPTG and purified by affinity chromatography with nickel-nitrilotriacetic acid (Ni-NTA) resin (Qiagen, Valencia, Calif.), according to the manufacturer's protocol. The purified recombinant protein was dialyzed in PBS to remove the denaturant and used to produce polyclonal antibodies in mice following standard procedures.

### Example 9

### Cell Migration Assay

Directional cell migration was assayed using a previously described method (19, 20). In brief, equal numbers of CL1-0 cells stably transfected with an empty vector or YY1 (68) were cultured to confluence as described in Example 6 in 6-well plates, at which time the monolayer was scraped with a cell scraper (Costar®, Acton, MA) to create a 3-mm track devoid of cells in the center of the chamber. Resulting cellular debris was removed by washing with PBS. These wound tracks were washed to remove detached cells, and fresh medium was added. Cells were incubated in RPMI medium with 10% serum and appropriate antibiotics, stained with crystal violet, then photographed at 0, 24, 48, and 72 hr after wounding. Cells that migrated across the regions of the wound edge were counted as migratory.

### Example 10

### Microarray Analysis

Microarrays containing 9,600 PCR-amplified cDNA fragments were prepared on nylon membranes by an arraying machine as previously described (13, 57). The 9600 nonredundant EST clones were Integrated Molecular Analysis of Genomes and their Expression (IMAGE) human cDNA clones, each representing a putative gene cluster with an assigned gene name in the Unigene clustering system (56). Hybridization experiments were performed in triplicate. Briefly, mRNA derived from each lung cancer cell line was labeled with biotin during reverse transcription. The biotin-labeled cDNA was used as a probe and hybridized with the microarray membranes. Probe preparation, hybridization, and color development were also performed as described previously (13, 57).

As shown in FIG. 9, HLJ1 expression levels correlated negatively with the invasive capacity of the lung cancer cell lines. The cells with the least metastic potential, CL1-0, expressed the most HLJ1 and the most highly metastatic cells, CL1-5-F4, expressed the least HLJ1 (68).

### Example 11

### Real-Time Quantitative RT-PCR (RTQ RT-PCR)

HLJ1 mRNA expression was quantified by real-time quantitative RT-PCR (RTQ RT-PCR) using TBP as an internal control. The primers, probes, and detailed procedures have been described previously (18). Briefly, each amplification mixture containing 10 ng of total RNA was subjected to one cycle of reverse transcription and 40 cycles of the polymerase chain reaction. All experiments were performed in triplicate. The relative expression level of HLJ1 compared to TBP was defined as −ΔCT=−[CT_{HLJ}1−CT_{TBP}]. The HLJ1 mRNA/TBP mRNA ratio was calculated as 2^{-ΔCT}×K (K: constant).

Total RNA was extracted from resected cancer tissue using an RNA extraction kit using TRIzol (Invitrogen Corp., Carlsbad, Calif.) according to manufacturer's instructions. The primers were based on the cDNA Sequence forward primer QHLJ1-F=5′-CCAGC AGACATTGTTTTATCATT-3′ (SEQ. ID. NO.:42); and reverse primer QHLJ1-R=5′-CCATCCAGTGTTGGTACATTAATT-3′(SEQ. ID. NO.:39). The sequence of the probe used to detect and quantify the RT-PCR product was 5′-ATTAGTTTACGAGAGGCATTGTGTGGC (SEQ. ID. NO.:43).

The primers and probes used for quantitative RT-PCR of the internal control, TATA-box binding protein (TBP) mRNA, (GenBank accession no. X54993) have been previously described (59). Each assay included a standard curve, a no-template control, and triplicate total RNA samples. The reaction conditions were as previously described (60). The fluorescence emitted by the reporter dye was detected on-line in real-time using the ABI prism 7700 Sequence detection system (PE Applied Biosystem, Foster City, Calif.). The amount of HLJ-1 cDNA relative to the amount of TBP cDNA was measured as −ΔCT=−[CT_{HLJ}1−CT_{TBP}]. The ratio of HLJ-1 mRNA copies relative to TBP mRNA copies was defined as 2^{-ΔCT}×K, where K is a constant.

### Example 12

### Subcellular Localization of HLJ1

Indirect immunofluorescence was performed by seeding CL1-5 cells in four-well chamber sides (Iwaki, Japan). After they reached 80% confluence, the cells were rinsed with PBS and fixed with 4% paraformaldehyde, permeabilized with 0.2% Triton X-100, and incubated at room temperature for 1 h with primary antibodies, namely mouse polyclonal antibodies to HLJ1 and rabbit polyclonal antibodies to nucleolin, a protein selectively localized to the nucleolus. Staining was performed with FITC-conjugated anti-mouse IgG antibody, rhodamine-conjugated anti-rabbit IgG antibody (Santa Cruz Biotechnology, Inc., Santa Cruz, Calif., USA) and DAPI (1 μg/ml) (Vector, Burlingame, Calif., USA). Images were viewed and collected with a confocal fluorescence microscope.

### Example 13

### Cell Proliferation Assay

Cells were seeded onto 96-well plates at 4000 cells per well in culture media (100 μl). After culturing for various times, cell numbers were measured by 3-(4,5-dimethylthiazol-2-yl)-2,5-diphenyltetrazolium bromide (MTT) assay according to the protocol provided by Roche Molecular Biochemicals. In the MTT assay, 10 μl of the MTT solution (5 mg/ml) were added to each well and the cells cultured for 4 h at 37° C. One hundred μl of 0.04 N HCl in isopropanol were then added to each well, and mixed vigorously to solubilize colored crystals produced within the cells. Color absorbance at 570 nm compared to reference wave absorbance at 630 nm was measured by a multiwell scanning spectrophotometer.

### Example 14

### Anchorage-Independent Growth

Anchorage independent growth was assayed by the ability of cells to grow in soft agar. The assay medium was composed of a bottom layer of 0.6% agarose in RPMI 1640, and a top layer of 0.3% agarose (soft agar). Cells from stable transfectants and controls were seeded at a density of 2,000 cells per well in a 6-well plate in triplicate. The plates were incubated at 37° C. with 5% CO₂ for 2 weeks and then stained with crystal violet. Colonies greater than 1 mm were counted under an inverted microscope. Colony formation was assessed in 3 independent experiments.

### Example 15

### In Vitro Invasion Assay

In vitro Matrigel™ invasion assays were performed in 6.5-mm transwell chambers (8 μm pore size; Costar®). The transwell filters in the chambers were coated with Matrigel™ (Becton Dickinson, Franklin Lakes, N.J.) (58). After a 16 or 24 h incubation, the filter was gently removed from the chamber, and the noninvasive cells on the upper surface were removed by wiping with a cotton swab. The cells that invaded the Matrigel™ and attached to the lower surface of the filter were fixed with methanol and stained with Giemza solution (Sigma, St. Louis, Mo.). The number of cells attached to the lower surface of the polycarbonate filter was counted under a light microscope at 200× magnification. Five fields of adherent cells were randomly counted in each well and the results were numerically averaged. Assays were performed in triplicate.

### Example 16

### Tumorigenicity in SCID Mice

Six-week-old SCID mice were housed at a density of five mice per cage in an isolator and fed ad libitum with autoclaved food. To determine tumor growth in animals, cells were trypsinized, washed, centrifuged, and re-suspended in Hank's balanced salt solution HBSS (Life Technologies, Inc., Gaithersburg, Md.). A total volume of 0.2 ml containing 5×10⁶ cells was subcutaneously injected into the dorsal region of each animal. Injected mice were examined every 5 or 7 days for tumor appearance and tumor volumes were estimated from the product of the three perpendicular diameters. After 28 days, animals were sacrificed and tumors were weighed. Nodules were confirmed to be malignant by histological examination.

### Example 17

### Human Tumor Specimens

Lung adenocarcinoma tumor tissue sections from 43 patients, including 26 men and 17 women (mean age±standard deviation =64.7±10.9), treated at the National Taiwan University Hospital between June 1994 and February 1999, were examined. None of the patients had received pre-operative adjuvant chemotherapy or radiation therapy. Specimens of lung cancer tissue and adjacent normal lung tissue obtained at surgery were snap-frozen in liquid nitrogen and stored at −80° C. until use. Post-surgical pathological staging classified the 43 tumors as 17 stage 1, 5 stage 11,17 stage III, and 4 stage 1V according to the international “Tumor Nodes Metastasis” (TNM) classification. Metastasis to the lymph nodes or other organs had occurred in 23 of the 43 patients.

### Example 18

### Screening for Modulators of HLJ1

A candidate modulator substance is applied to cells, inter alia tumor cells and normal control cells, using cell culture methods as described in Example 6 and other cell maintenance methods known in the art. The cells may express high or low levels of HLJ1, either naturally, by enhancing the amount of HLJ1 expression, e.g., by transfection with HLJ1 DNA, or by modulating the level of HLJ1 transcription or translation, or by decreasing the amount of HLJ1 expression, e.g., by inhibitory RNA techniques known in the art, or the use of knockout animals, as known in the art. Screening can be performed on cells in vivo, ex vivo, and in vitro. Candidate modulators can specifically bind to components on the surface or inside the cells, and polynucleotides or polypeptides, or otherwise specifically modulate biological activity. The biological activity can be measured using any assay known in the art.

Suitable candidate modulators include agonists, antagonists, antibodies, small molecule drugs, soluble receptors, natural ligands, and peptide aptamers, They also include agents known to modulate intracellular pathways that regulate transcription, e.g., agents that modulate ligands, receptors, signal transduction molecules, and/or transcription factors.

### Example 19

### Statistical Analysis

Experiments were performed in triplicate and analyzed by ANOVA (Excel, Microsoft; Taipei, Taiwan, Republic of China) for significant differences. P values<0.05 were considered statistically significant. Where appropriate, the data are presented as the mean±SD.

Disease survival and recurrence can be estimated by non-parametric methods, e.g., as performed herein, the Kaplan-Meier method, which estimates the probability of survival or recurrence over time (72). Results of the Kaplan-Meier are shown in FIG. 16. Disease survival and recurrence can also be estimated by parametric methods, or by stratification of the data by dividing it into subsamples based on one or more characteristics of the population.

### REFERENCES

The specification is most thoroughly understood in light of the cited references, all of which are hereby incorporated by reference in their entireties. The disclosures of the patents, applications, and other references cited above are also herein incorporated by reference in their entireties. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.
1. Welch, W. J. Mammalian stress response: cell physiology, structure/function of stress proteins, and implications for medicine and disease. Physiol. Rev. (1992) 72:1063-1081.
2. Hendric, J. P., and Hartl, F.-U. Molecular chaperone functions of heat-shock proteins. Annu. Rev. Biochem. (1993) 62:349-384.
3. Craig, E. A., Weissman, J. S., and Horwich, A. L. Heat shock proteins and molecular chaperones: mediators of protein conformation and turnover in the cell. Cell (1994) 78:365-372.
4. Caroline, J. and Richard, I. M. Role of the heat shock response and molecular chaperons in oncogenesis and cell death. J. Natl. Cancer Inst. (2000) 92:1564-1572.
5. Hartl, F.-U. Molecular chaperones in cellular protein folding. Nature (1996) 381:571-580.
6. Gottesman, S., Wickner, S., and Maurizi, M. R. Protein quality control: triage by chaperones and proteases. Genes Dev. (1997) 11:815-823.
7. Hoe, K. L., Won, M., Chung, K. S., Jang, Y. J., Lee, S. B., Kim, D. U., Lee, J. W., Yun, J. H., and Yoo, H. S. Isolation of a new member of DnaJ-like heat shock protein (Hsp40) from human liver. Biochim. Biophys. Acta (1998) 1383:4-8.
8. Ohtsuka, K. and Hata, M. Mammalian HSP40/DNAJ homologs: cloning of novel cDNAs and a proposal for their classification and nomenclature. Cell Stress Chaperones. (2000) 5:98-112.
9. Hattori, H., Liu, Y.-C., Tohnai, I., Ueda, M., Kaneda, T., Kobayashi, T., Tanabe, K., and Ohtsuka, K. Intracellular localization and partial amino acid. Sequence of a stress-inducible 40 kDa protein in HeLa cells. Cell Struct. Funct. (1992) 17:77-86.
10. Ohtsuka, K. Cloning of a cDNA for heat-shock protein hsp40, a human homologue of bacterial DNAJ. Biochem. Biophys. Res. Commun. (1993) 197:235-240.
11. Georgopoulos, C. The emergence of the chaperon machines. Trends Biochem. Sci. (1992) 17:295-299.
12. Hamajima, F., Hasegawa, T., Nakashima, I., and Isobe, K.-I. Genomic cloning and promoter analysis of the GAHSP40 gene. J. Cell. Biochem. (2002) 84:401-407.
13. Chen, J. J. W., Peck, K., Hong, T. M., Yang, S. C., Sher, Y. P., Shih, J. Y., Wu, R., Cheng, J. L., Roffler, S. R., Wu, C. W., and Yang, P. C. Global analysis of gene expression in invasion by a lung cancer model. Cancer Res. (2001) 61:5223-5230.
14. Quandt, K., Frech, K., Karas, H., Wingender, E., and Werner, T. Matlnd and MatInspector: new fast and versatile tools for detection of consensus matches in nucleotide sequence data. Nucleic Acids Res. (1995) 23:4878-4884.
15. Prestridge, D. S. SIGNAL SCAN: A computer program that scans DNA sequences for eukaryotic transcriptional elements. CABIOS (1991) 7:203-206.
16. Heinemeyer, T., Chen, X., Karas, H., Kel, A. E., Kel, O. V., Liebich, I., Meinhardt, T., Reuter, I., Schacherer, F., and Wingender, E. Expanding the TRANSFAC database towards an expert system of regulatory molecular mechanisms. Nucleic Acids Res. (1999) 27: 318-322.
17. Matz, M., Shagin, D., Bogdanova, E., Britanova, O., Lukyanov, S., Diatchenko, L., and Chenchik, A. Amplification of cDNA ends based on template-switching effect and step-out PCR. Nucleic Acids Res. (1999) 27:1558-1560.
18. Chen, J. J. W., Yao, P. L., Yuan, A., Hong, T. M., Shun, C. T., Kuo, M. L., Lee, Y. C., and Yang, P. C. Up-Regulation of tumor interleukin-8 expression by infiltrating macrophages: its correlation with tumor angiogenesis and patient survival in non-small cell lung cancer. Clin. Cancer Res. (2003) 9:729-737.
19. Michael, V. A., Sheri, E. K., and Wendt, K. W. AIF-1 is an actin-polymerizing and Rac1-activating protein that promotes vascular smooth muscle cell migration. Circ Res. (2003) 92:1107-1114.
20. Iijima, K., Yoshizumi, M., Hashimoto, M., Akishita, M., Kozaki, K., Ako, J., Watanabe, T., Ohike, Y., Son, B., Yu, J., Nakahara, K., and Ouchi, Y. Red wine polyphenols inhibit vascular smooth muscle cell migration through two distinct signaling pathways. Circulation (2002) 105:2404-2410.
21. Mantovani, R. A survey of 178 NF-Y binding CCAAT boxes. Nucleic Acids Res. (1998) 26:1135-1143.
22. Bird, A. P. CpG rich islands and the function of DNA methylation. Nature (1986) 321: 209-213.
23. Galvin, K. M., and Shi, Y. Multiple mechanisms of transcriptional repression by YY1. Mol. Cell Biol. (1997) 17:3723-3732.
24. Shi, Y., Seto, E., Chang, L. S., and Shenk, T. Transcriptional repression by YY1, a human GLI-Kruppel-related protein, and relief of repression by adenovirus E1A protein. Cell (1991) 67:377-388.
25. Ficzycz, A., Eskiw, C., Meyer, D., Marlry, K. E., Hurt, M., and Ovsenek, N. Expression, activity, and subcellular localization of the Ying Yang 1 transcription factor in Xenopus oocytes and embryos. J. Biol. Chem. (2001) 276:22,819-22,825.
26. Ficzycz, A., and Ovsenek, N. The Ying Yang 1 transcription factor associates with ribonucleoprotein (mRNP) complexes in the cytoplasm of Xenopus oocytes. J. Biol. Chem. (2002) 277:8382-8387.
27. Riggs, K. J., Saleques, S., Wong, K. K. Merrell, K. T., Lee, J. S., Shi, Y., and Calame, K. Ying-yang 1 activates the c-myc promoter. Mol. Cell Biol. (1993) 13:7487-7495.
28. Lee, J. S., Zhang, X., and Shi, Y. Differential interactions of the CREB/ATF family of transcription factors with p300 and adenovirus E1A. J. Biol. Chem. (1996) 271:17,666-17,674.
29. Yao, Y. L., Yang, W. M., and Seto, E. Regulation of transcription factor YY1 by acetylation and deacetylation. Mol. Cell Biol. (2001) 21:5979-5991.
30. Furlong, E. E., Rein, T., and Martin, F. YY1 and NF1 both activate the human p53 promoter by alternatively binding to a composite element, and YY1 and E1A cooperate to amplify p53 promoter activity. Mol. Cell Biol. (1996) 16:5933-3945.
31. Usheva, A., and Shenk, T. TATA-binding protein-independent initiation: YY1, TFIIB and RNA polymerase 11 direct basal transcription on supercoiled template DNA. Cell (1994) 76:1115-1121.
32. Crowe, D. L., and Brown, T. N. Transcriptional inhibition of matrix metalloproteinase 9 (MMP-9) activity by a c-fos/estrogen receptor fusion protein is mediated by the proximal AP-1 site of the MMP-9 promoter and correlates with reduced tumor cell invasion. Neoplasia (1999) 1:368-372.
33. von Marschall, Z., Scholz, A., Cramer, T., Schafer, G., Schirner, M., Oberg, K., Wiedenmann, B., Hocker, M., and Rosewicz, S. Effects of interferon alpha on vascular endothelial growth factor gene transcription and tumor angiogenesis. J. Natl. Cancer Inst. (2003) 95:437-448.
34. Chen, H. W., Chien, C. T., Yu, S. L., Lee, Y. T., and Chen, W. J. Cyclosporine A regulate oxidative stress-induced apoptosis in cardiomyocytes: mechanisms via ROS generation, iNOS and Hsp70. Br. J. Pharmacol. (2002) 137:771-781.
35. Shih, J. Y., Yang, S.C., Hong, T. M., Yuan, A., Chen, J. J. W., Yu, C. J., Chang, Y. L., Lee, Y. C., Peck, K., Wu, C. W., and Yang, P. C. Collapsin response mediator protein-1 and the invasion and metastasis of cancer cells. J. Natl. Cancer Inst. (2001) 93:1392-1400.
36. Parkin, D. M., Bray, F. I., and Devesa, S S. Cancer burden in the year 2000. The global picture. Eur. J. Cancer. (2001) 37(Suppl 8): S4-66.
37. Department of Health, Executive Yuan, Taiwan, R.O.C. Taiwan area main causes of death (2002). Health and Vital Statistics (http://www.doh.gov.tw.)
38. Hoffman, P. C., Mauer, A. M., and Vokes, E. E. Lung cancer. Lancet (2000) 355(9202):479-85.
39. Jemal, A., Thomas, A, Murray, T., and Thun, M. Cancer statistics 2002 CA Cancer J. Clin. (2002) 52(1):23-47.
40. Jaklitsch, M. T., Strauss, G. M., Healey, E. A., DeCamp, M. M. Jr., Liptay, M. J., and Sugarbaker, D. J. An historical perspective of multi-modality treatment for resectable non-small cell lung cancer. Lung Cancer (1995) 12 (Suppl 2): S17-32.
41. Niklinski, J., Niklinska, W., Chyczewski, L., Becker, H. D, and Pluygers, E. Molecular genetic abnormalities in premalignant lung lesions: biological and clinical implications. Eur. J. Cancer Prev. (2001) 10(3):213-226.
42. Mountain, C.F. Revisions in the international system for staging lung cancer. Chest (1997) 111(6):1710-1717.
43. Meyer, T., and Hart, I. R. Mechanisms of tumour metastasis. Eur. J. Cancer. (1998) 34: 214-221.
44. Yoneda, T. Cellular and molecular mechanisms of breast and prostate cancer metastasis to bone. Eur. J. Cancer. (1998) 34:240-245.
45. Zhao, H., Jhanwar-Uniyal, M., Datta, P. K., Yemul, S., Ho, L., Khitrov, G., Kupershmidt, I. Pasinetti, G. M., Ray, T., Athwal, R. S., and Achary, M. P. Expression profile of genes associated with antimetastatic gene: nm23-mediated metastasis inhibition in breast carcinoma cells. Int. J. Cancer (2004) 109(1):65-70.
46. Ouatas, T., Salerno, M., Palmieri, D., and Steeg, P.S. Basic and translational advances in cancer metastasis: Nm23. J. Bioenerg. Biomembr. (2003) 35(1):73-79.
47. Nicolson, G. L., Nawa, A., Toh, Y., Taniguchi, S., Nishimori, K., and Moustafa, A. Tumor metastasis-associated human MTA1 gene and its MTA1 protein product: role in epithelial cancer cell invasion, proliferation and nuclear regulation. Clin. Exp. Metastasis (2003) 20(1):19-24.
48. Gao, A. C., Lou, W., Dong, J. T., and Isaacs, J. T. CD44 is a metastasis suppressor gene for prostatic cancer located on human chromosome 11 p13. Cancer Res. (1997) 57(5):846-849.
49. Shiomi, T., and Okada, Y. MT1-MMP and MMP-7 in invasion and metastasis of human cancers. Cancer Metastasis Rev. (2003) 22(2-3):145-152.
50. Lee, J. H., Seo, Y. W., Park, S.R., Kim, Y. J., and Kim, K.K. Expression of a splice variant of KAI1, a tumor metastasis suppressor gene, influences tumor invasion and progression. Cancer Res. (2003) 63(21):7247-7255.
51. Jiang, W. G. E-cadherin and its associated protein catenins, cancer invasion and metastasis. Br. J. Surg. (1996) 83(4):437-446.
52. Bremnes, R. M., Veve, R., Hirsch, F. R., and Franklin, W. A. The E-cadherin cell-cell adhesion complex and lung cancer invasion, metastasis, and prognosis. Lung Cancer (2002) 36(2):115-124.
53. Harms, J. F, Welch, D. R., and Miele, M. E. KISS1 metastasis suppression and emergent pathways. Clin. Exp. Metastasis (2003) 20(1):11-18.
54. Cheetham, M. E. and Caplan, A. J. Structure, function and evolution of DnaJ: conservation and adaptation of chaperone function. Cell Stress Chaperones (1998) 3(1):28-36.
55. Chu, Y. W., Yang, P. C., Yang, S. C., Shyu, Y. C., Hendrix, M. J., Wu, R., and Wu, C. W. Selection of invasive and metastatic subpopulations from a human lung adenocarcinoma cell line. Am. J. Respir. Cell Mol. Biol. (1997) 17(3):353-360.
56. Schuler, G. D. Pieces of the puzzle: expressed sequence tags and the catalog of human genes. J. Mol. Med. (1997) 75(10):694-698.
57. Hong, T. M., Yang, P. C., Peck, K., Chen, J. J., Yang, S.C., Chen, Y. C., and Wu, C. W. Profiling the downstream genes of tumor suppressor PTEN in lung cancer cells by complementary DNA microarray. Am. J. Respir. Cell Mol. Biol. (2000) 23(3):355-363.
58. Albini, A., Iwamoto, Y., Kleinman, H. K., Martin, G. R., Aaronson, S. A., Kozlowski, J. M., and McEwan, R.N. A rapid in vitro assay for quantitating the invasive potential of tumor cells. Cancer Res. (1987) 47(12):3239-3245.
59. Bieche, I., Onody, P., Laurendeau, I, Olivi M, Vidaud D, Lidereau R, and Vidaud M. Real-time reverse transcription-PCR assay for future management of ERBB2-based clinical applications. Clin. Chem. (1999) 45(8 Pt 1):1148-1156.
60. Yuan, A., Yang, P. C., Yu, C. J., Chen, W. J., Lin, F. Y., Kuo, S. H. and Luh K. T. Interleukin-8 messenger ribonucleic acid. expression correlates with tumor progression, tumor angiogenesis, patient survival and timing of relapse in non-small-cell lung cancer. Am. J. Respir. Crit. Care Med. 162:1957-1963.
61. Izawa, I., Nishizawa, M., Ohtakara, K., Ohtsuka, K., Inada, H., and Inagaki, M. Identification of Mrj, a DnaJ/Hsp40 family protein, as a keratin 8/18 filament regulatory protein. J. Biol. Chem. (2000) 275(44):34,521-34,527.
62. Sedbrook, J. C., Chen, R., and Masson, P. H. ARG1 (altered response to gravity) encodes a DnaJ-like protein that potentially interacts with the cytoskeleton. Proc. Natl. Acad. Sci. (1999) 96:1140-1145.
63. Kurzik-Dumke, U., Gundacker, D., Renthrop, M., and Gateff, E. Tumor suppression in Drosophila is causally related to the function of the lethal (2) tumorous imaginal discs gene, a dnaJ homolog. Dev. Genet (1995) 16(1):64-76.
64. Sullivan, C. S. and Pipas, J. M. T antigens of simian virus 40: molecular chaperones for viral replication and tumorigenesis. Microbiol. Mol. Biol. Rev. (2002) 66(2): 179-202.
65. Schilling, B., De-Medina, T., Syken, J., Vidal, M., and Munger, K. A novel human DnaJ protein, hTid-1, a homolog of the Drosophila tumor suppressor protein Tid56, can interact with the human papillomavirus type 16 E7 oncoprotein. Virology (1998) 247(1):74-85.
66. Canamasas, I., Debes, A., Natali, P. G., and Kurzik-Dumke, U. Understanding human cancer using Drosophila: Tid47, a cytosolic product of the DnaJ-like tumor suppressor gene 12Tid, is a novel molecular partner of patched related to skin cancer. J. Biol. Chem. (2003) 278(33):30,952-30,960.
67. Kelley, W. L. The J-domain family and the recruitment of chaperone power. Trends Biochem. Sci. (1998) 23(6):222-227.
68. Wang, C.-C, Tsai, M.-F, Hong, T.-M., Chang, G.-C, Chen, C.-Y, Yang, W.-M, Chen, J. J. W., and Yang, P.-C. The transcriptional factor YY1 upregulates the novel invasion suppressor HLJ1 expression and inhibits cancer cell invasion. Oncogene (2005) 24:4081-4093.
69. Altschul, S.F., Gish, W., Miller, W., Myers, E.W., and Lipman, D. J. Basic local alignment search tool. J. Mol. Biol. (1990) 215:403-410.
70. Uberbacher, E. C., and Mural, R. J. Locating protein-coding regions in human DNA sequences by a multiple sensor-neural network approach. Proc. Natl. Acad. Sci. USA (1991) 88:11261-11265.
71. Jones, J., Field, J. K., and Risk, J. M. A comparative guide to gene prediction tools for the bioinformatics amateur. Int. J. Oncol. (2002) 20:697-705.
72. Kaplan, E. L. and Meier, P. Nonparametric estimation from incomplete observations. J. Amer. Statistical Assoc. (1958) 53:457-448.

In accordance with an objective of the present invention, there is provided:

1. An isolated nucleic acid molecule comprising the 5′ regulatory region of the human HLJ1 gene, comprising nucleotides −2126 to +17 of the human HLJ1 gene, or one or more fragments or variants thereof.

2. The isolated nucleic acid molecule of claim 1, further comprising a transcriptional start site located 176 bp upstream of the translation initiation site.

3. The isolated nucleic acid molecule of claim 1, further comprising transcription factor binding sites comprising one or more YY1 binding sites selected from YY1 binding sites located at nucleotides −232 to −228, −211 to −207, −185 to −181; and −154 to −151.

4. The isolated nucleic acid molecule of claim 1, further comprising one or more of
(a) (a) an enhancer region at nucleotides −2126 to −1039;
(b) (b) a silencing element at nucleotides −1,255 to −1,039; and
(c) (c) a GC box beginning at nucleotide −761.

5. The nucleic acid molecule of claim 1, wherein the nucleic acid molecule is operably linked to a structural gene.

6. The nucleic acid molecule of claim 5, wherein the structural gene is a reporter gene.

7. The nucleic acid molecule of claim 6, wherein the reporter gene is luciferase.

8. A vector comprising the nucleic acid molecule of claim 1.

9. A host cell transfected with the nucleic acid molecule of claim 1.

10. The host cell of claim 9, wherein the host cell is a cancer cell.

11. The host cell of claim 10, wherein the cancer cell is an adenocarcinoma cell.

12. The host cell of claim 11, wherein the adenocarcinoma cell comprises a cell line.

13. The host cell of claim 12, wherein the cell line is chosen from CL1-0, CL1-1, CL1-5, CL1-5-F4, CL1-5/HLJ1, PCC1, PCY3-1, PCY4-2, and PCY4-5.

14. The host cell of claim 11, wherein the adenocarcinoma cell is a lung cell.

15. The host cell of claim 14, wherein the lung adenocarcinoma cell is human.

16. An HLJ1 core promoter region comprising nucleotides −232 to +176 of the HLJ1 gene.

17. An isolated nucleic acid molecule comprising the nucleotide sequence selected from SEQ. ID. NOS.:1-13, a fragment of any of these, and a variant of any of these.

18. A single stranded oligonucleotide comprising a sequence selected from SEQ. ID. NO.:1, SEQ. ID. NO.:2, SEQ. ID. NO.:3, SEQ. ID. NO.:4, SEQ. ID. NO.:5, SEQ. ID. NO.:6, SEQ. ID. NO.:7, SEQ. ID. NO.:8, SEQ. ID. NO.:9, SEQ. ID. NO.:10, SEQ. ID. NO.:11, SEQ. ID. NO.:12, and SEQ. ID. NO.:13.

19. A method of identifying a compound that modulates HLJ1 gene expression, the method comprising:
(a) providing a cell transfected with the nucleic acid molecule of claim 17;
(b) contacting the cell with a test compound; and
(c) determining the level of expression of the EGFP gene in the presence of the test compound,
wherein a low level of expression of EGFP is an indication that the test compound inhibits the promoter activity of the HLJ1 gene.

20. A method of determining the metastatic ability of a cell of unknown metastatic ability comprising:
(a) providing a cell of unknown metastatic ability;
(b) determining its level of HLJ1 gene expression; and
(c) comparing the HLJ1 expression level of the cell with unknown metastatic ability with a positive control of known metastatic ability and a negative control non-metastatic cell;
wherein the expression level negatively correlates with metastatic ability.

21. A method of decreasing the metastatic ability of a cell possessing such ability, comprising:
(a) providing a metastatic cell; and
(b) increasing the expression of the HLJ1 gene in the cell;
wherein HLJ1 gene expression negatively correlates with the metastatic ability of the cell.

22. The method of claim 21, wherein the cell is a cancer cell.

23. The method of claim 22, wherein the cancer cell is a human lung adenocarcinoma cell.

24. The method of claim 21, further comprising transfecting the cell with a nucleic acid molecule corresponding to the human HLJ1 gene, a regulatory fragment, or a variant thereof, wherein expressing the nucleic acid molecule increases HLJ1 gene expression.

25. The method of claim 24, wherein the cell is a cancer cell.

26. The method of claim 25, wherein the cancer cell is a human lung adenocarcinoma cell.

27. A method of inhibiting cell proliferation, comprising:
(a) providing a proliferating cell; and
(b) increasing the expression of the HLJ1 gene in the cell;
wherein HLJ1 gene expression inhibits the proliferation of the cell.

28. The method of claim 27, wherein the cell is a cancer cell.

29. The method of claim 28, wherein the cancer cell is a human lung adenocarcinoma cell.

30. The method of claim 27, further comprising transfecting the cell with a nucleic acid molecule corresponding to the human HLJ1 gene, a regulatory fragment, or a variant thereof, wherein expressing the nucleic acid molecule increases HLJ1 gene expression.

31. A method of decreasing the invasive ability of a cell possessing such ability, comprising:
(a) providing an invasive cell; and
(b) increasing the expression of the HLJ1 gene in the cell;
wherein HLJ1 gene expression inhibits the invasive ability of the cell.

32. The method of claim 31, wherein the cell is a cancer cell.

33. The method of claim 32, wherein the cancer cell is a human lung adenocarcinoma cell.

34. The method of claim 31, further comprising transfecting the cell with a nucleic acid molecule corresponding to the human HLJ1 gene, a regulatory fragment, or a variant thereof, wherein expressing the nucleic acid molecule increases HLJ1 gene expression.

35. A method of diagnosing cancer comprising:
(a) providing a mammalian tissue sample; and
(b) determining the level of HLJ1 gene expression in comparison to non-malignant control tissue;
wherein the level of HLJ1 gene expression indicates a diagnosis of cancer.

36. The method of claim 35, wherein the cancer is lung adenocarcinoma.

37. A method of predicting the quantitative probability of surviving lung cancer or of avoiding a recurrence of lung cancer in a patient diagnosed as having lung cancer comprising:
(a) measuring the expression of HLJ1 mRNA in the cancer cells of the patient; and
(b) applying a statistical method of analysis to estimate the probability of survival over time;
wherein the statistical method predicts survival or recurrence.

38. A therapeutic composition comprising a modulator of HLJ1, and a pharmaceutically acceptable carrier.

39. A method of treating a patient in need of such treatment with the composition of claim 38 comprising:
(a) providing the composition of claim 38; and
(b) administering the composition in a manner chosen from orally, parenterally, by implantation, by inhalation, mucosally, intranasally, intravenously, intra-arterially, intracardiacally, subcutaneously, intradermally, intraperitoneally, transdermally, intraventricularly, intracranially, and intrathecally;
wherein administering the composition treats the patient.

40. A method of treating a lung adenocarcinoma patient comprising:
(a) transfecting one or more adenocarcinoma cell of the patient with a construct that increases the promoter activity of HLJ1; and
(b) increasing the expression of HLJ1 in one or more adenocarcinoma cell of said patient;
wherein increasing the expression treats the patient.
